(19) United States
(12) Patent Application Publication
(10) Pub. No.: US 2007/0191414 A1
(43) Pub. Date: Aug. 16, 2007

(54) Novel isoamido-substituted hydroxy-6-phenylphenanthridines

(75) Inventor: Ulrich Kautz, Allensbach (DE)

Correspondence Address: NATH & ASSOCIATES PLLC, 112 South West Street, Alexandria, VA, 22314, US

(73) Assignee: Altana Pharma AG, Konstanz (DE)

(21) Appl. No.: 10/591,478

(22) PCT Filed: Mar. 8, 2005
(86) PCT No.: PCT/EP05/51025

§ 371 Date: Sep. 27, 2006

(30) Foreign Application Priority Data
Mar. 9, 2004 (EP) 04100959.8
Jan. 27, 2005 (EP) 05100545.2

(51) Int. Cl. A61K 31/4741; A61K 31/473; C07D 221/12

(52) U.S. Cl. 514/285; 514/298; 546/62; 546/109

(57) ABSTRACT

Compounds of the formula I 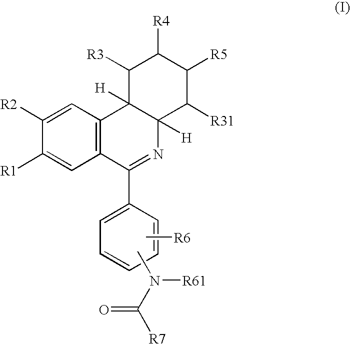 in which the substituents have the definitions provided in the specification, are novel, effective PDE4 inhibitors.

### FIELD OF APPLICATION OF THE INVENTION

The invention relates to novel isoamido-substituted hydroxy-6-phenylphenanthridine derivatives, which are used in the pharmaceutical industry for the production of pharmaceutical compositions.

### Known Technical Background

The International Patent applications WO99/57118 and WO02/05616 describe 6-phenylphenanthridines as PDE4 inhibitors.

In the International Patent application WO99/05112 substituted 6-alkylphenanthridines are described as bronchial therapeutics.

In the European Patent application EP 0490823 dihydroisoquinoline derivatives are described which are useful in the treatment of asthma.

In the International Patent application WO97/35854 phenanthridines substituted in the 6-position are described as bronchial therapeutics.

The International Patent applications WO2004/019944 and WO2004/019945 disclose hydroxy-substituted 6-phenylphenanthridines as PDE4 inhibitors.

### DESCRIPTION OF THE INVENTION

It has now been found that the novel isoamido-substituted 2- or 3-hydroxy-6-phenylphenanthridines described in greater detail below differ from the previously known compounds by unanticipated and sophisticated structural alterations and have surprising and particularly advantageous properties.

The invention thus relates to compounds of formula I, 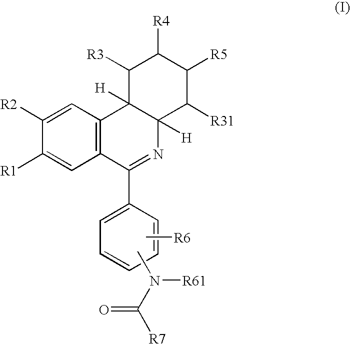
in which
R1 is hydroxyl, 1-4C-alkoxy, 3-7C-cycloalkoxy, 3-7C-cycloalkylmethoxy, 2,2-difluoroethoxy, or completely or predominantly fluorine-substituted 1-4C-alkoxy,
R2 is hydroxyl, 1-4C-alkoxy, 3-7C-cycloalkoxy, 3-7C-cycloalkylmethoxy, 2,2-difluoroethoxy, or completely or predominantly fluorine-substituted 1-4C-alkoxy,
   or in which
R1 and R2 together are a 1-2C-alkylenedioxy group,
R3 is hydrogen or 1-4C-alkyl,
R31 is hydrogen or 1-4C-alkyl,
   either, in a first embodiment (embodiment a) according to the present invention,
R4 is —O—R41, in which
R41 is hydrogen, 1-4C-alkyl, 1-4C-alkoxy-1-4C-alkyl, hydroxy-2-4C-alkyl, 1-7C-alkylcarbonyl, or completely or predominantly fluorine-substituted 1-4C-alkyl, and
R5 is hydrogen or 1-4C-alkyl,
   or, in a second embodiment (embodiment b) according to the present invention,
R4 is hydrogen or 1-4C-alkyl, and
R5 is —O—R51, in which
R51 is hydrogen, 1-4C-alkyl, 1-4C-alkoxy-1-4C-alkyl, hydroxy-24C-alkyl, 1-7C-alkylcarbonyl, or completely or predominantly fluorine-substituted 1-4C-alkyl,
R6 is hydrogen, halogen, 1-4C-alkyl or 1-4C-alkoxy,
R61 is hydrogen, 1-4C-alkyl or 1-4C-alkoxy-2-4C-alkyl,
R7 is Het1, Har1, 3-7C-cycloalkyl, or 1-4C-alkyl substituted by R8, in which
Het1 is optionally substituted by R71 and is a monocylic 3- to 7-membered fully saturated heterocyclic ring radical,
which is bonded via a ring carbon atom to the carbonyl moiety of the —C(O)N(R61)- group, and
which comprises one nitrogen atom and optionally one further heteroatom selected from the group consisting of nitrogen, oxygen and sulfur, and, optionally,
to which ring one or two oxo groups are bonded, in which
R71 is 1-4C-alkyl, or completely or partially fluorine-substituted 1-4C-alkyl,
Har1 is optionally substituted by R72 and/or R73, and is a 5- or 6-membered monocyclic unsaturated heteroaryl radical comprising 1 to 4 heteroatoms selected independently from the group consisting of oxygen, nitrogen and sulfur, in which
R72 is halogen, 1-4C-alkyl, 1-4C-alkoxy, 1-4C-alkoxy-2-4C-alkoxy, 1-4C-alkylthio, cyano, 1-4C-alkoxycarbonyl, carboxyl, hydroxyl, -A-N(R721)R722, pyridyl, or completely or partially fluorine-substituted 1-4C-alkyl, in which
A is a bond or 1-4C-alkylene,
R721 is hydrogen or 1-4C-alkyl,
R722 is hydrogen or 1-4C-alkyl,
or R721 and R722 together and with inclusion of the nitrogen atom, to which they are attached, form a heterocyclic ring Het2, in which
Het2 is optionally substituted by R723, and is a 3- to 7-membered saturated or unsaturated monocyclic heterocyclic ring radical comprising the nitrogen atom, to which R721 and R722 are bonded, and optionally one to three further heteroatoms independently selected from the group consisting of oxygen, nitrogen and sulfur, in which
R723 is 1-4C-alkyl,
R73 is halogen, 1-4C-alkoxy, 1-4C-alkoxy-2-4C-alkoxy, 1-4C-alkylthio, hydroxyl, amino or mono- or di-1-4C-alkylamino,
R8 is 1-4C-alkoxy, carbamoyl, carboxyl, 1-4C-alkoxycarbonyl, mono- or di-1-4C-alkylaminocarbonyl or —N(R81)R82, in which
R81 is hydrogen, 1-4C-alkyl, carbamoyl, amidino or 1-4C-alkylcarbonyl,
R82 is hydrogen or 1-4C-alkyl,
or R81 and R82 together and with inclusion of the nitrogen atom, to which they are attached, form a heterocyclic ring Het3, in which
Het3 is optionally substituted by R811, and is a 3- to 7-membered saturated monocyclic heterocyclic ring radical comprising the nitrogen atom, to which R81 and R82 are bonded, and optionally one further heteroatom selected from the group consisting of oxygen, nitrogen and sulfur, in which
R811 is 1-4C-alkyl,
   and the salts, the N-oxides and the salts of the N-oxides of these compounds.

1-4C-Alkyl represents a straight-chain or branched alkyl radical having 1 to 4 carbon atoms. Examples which may be mentioned are the butyl, isobutyl, sec-butyl, tert-butyl, propyl, isopropyl and preferably the ethyl and methyl radicals.

1-7C-Alkyl represents a straight-chain or branched alkyl radical having 1 to 7 carbon atoms. Examples which may be mentioned are the heptyl, isoheptyl (5-methylhexyl), hexyl, isohexyl (4-methylpentyl), neohexyl (3,3-dimethylbutyl), pentyl, isopentyl (3-methylbutyl), neopentyl (2,2-dimethylpropyl), butyl, isobutyl, sec-butyl, tert-butyl, propyl, isopropyl, ethyl or methyl radicals.

3-7C-Cycloalkyl represents cyclopropyl, cyclobutyl, cyclopentyl, cyclohexyl and cycloheptyl, of which cyclopropyl and cyclopentyl are preferred.

1-4C-Alkylene is a straight chain alkylene radical having 1 to 4 carbon atoms. Examples which may be mentioned in this context are the methylene (—CH₂—), ethylene (—CH₂—CH₂—), trimethylene (—CH₂—CH₂—CH₂—) and the tetramethylene (—CH₂—CH₂—CH₂—CH₂—) radical.

1-4C-Alkoxy represents radicals which, in addition to the oxygen atom, contain a straight-chain or branched alkyl radical having 1 to 4 carbon atoms. Examples which may be mentioned are the butoxy, isobutoxy, sec-butoxy, tert-butoxy, propoxy, isopropoxy and preferably the ethoxy and methoxy radicals.

3-7C-Cycloalkoxy represents cyclopropyloxy, cyclobutyloxy, cyclopentyloxy, cyclohexyloxy and cyclo-heptyloxy, of which cyclopropyloxy, cyclobutyloxy and cyclopentyloxy are preferred.

3-7C-Cycloalkylmethoxy represents cyclopropylmethoxy, cyclobutylmethoxy, cyclopentylmethoxy, cyclohexylmethoxy and cycloheptylmethoxy, of which cyclopropylmethoxy, cyclobutylmethoxy and cyclopentylmethoxy are preferred.

As completely or predominantly fluorine-substituted 1-4C-alkoxy, for example, the 2,2,3,3,3-pentafluoro-propoxy, the perfluoroethoxy, the 1,2,2-trifluoroethoxy, in particular the 1,1,2,2-tetrafluoroethoxy, the 2,2,2-trifluoroethoxy, the trifluoromethoxy and preferably the difluoromethoxy radicals may be mentioned. “Predominantly” in this connection means that more than half of the hydrogen atoms of the 1-4C-alkoxy radicals are replaced by fluorine atoms.

As completely or predominantly fluorine-substituted 1-4C-alkyl, for example, the 2,2,3,3,3-pentafluoropropyl, the perfluoroethyl, the 1,2,2-trifluoroethyl, in particular the 1,1,2,2-tetrafluoroethyl, the 2,2,2-trifluoroethyl, the trifluoromethyl and particularly the difluoromethyl radicals may be mentioned. “Predominantly” in this connection means that more than half of the hydrogen atoms of the 1-4C-alkyl radicals are replaced by fluorine atoms.

As completely or partially fluorine-substituted 1-4C-alkyl, for example, the 2,2,3,3,3-pentafluoropropyl, the perfluoroethyl, the 1,2,2-trifluoroethyl, the 1,1,2,2-tetrafluoroethyl, the 2,2,2-trifluoroethyl, the trifluoromethyl, the difluoromethyl and, in particular, the 2,2-difluoroethyl radicals may be mentioned.

1-2C-Alkylenedioxy represents, for example, the methylenedioxy [—O—CH₂—O—] and the ethylenedioxy [—O—CH₂—CH₂—O] radicals.

1-4C-Alkoxy-1-4C-alkyl represents one of the abovementioned 1-4C-alkyl radicals, which is substituted by one of the abovementioned 1-4C-alkoxy radicals. Examples which may be mentioned are the methoxy-methyl, the methoxyethyl and the isopropoxyethyl radicals, particularly the 2-methoxyethyl and the 2-isopropoxyethyl radicals.

1-4C-Alkoxy-2-4C-alkyl represents 2-4C-alkyl radicals, which are substituted by one of the abovementioned 1-4C-alkoxy radicals. Examples which may be mentioned are the methoxyethyl, ethoxyethyl and the isopropoxyethyl radicals, particularly the 2-methoxyethyl, 2-ethoxyethyl and the 2-isopropoxyethyl radicals.

1-7C-Alkylcarbonyl represents a radical which, in addition to the carbonyl group, contains one of the abovementioned 1-7C-alkyl radicals. Examples which may be mentioned are the acetyl, propionyl, butanoyl and hexanoyl radicals.

Hydroxy-2-4C-alkyl represents 2-4C-alkyl radicals, which are substituted by a hydroxyl group. Examples which may be mentioned are the 2-hydroxyethyl and the 3-hydroxypropyl radicals.

In addition to the nitrogen atom, mono- or di-1-4C-alkylamino radicals contain one or two of the abovementioned 1-4C-alkyl radicals. Di-1-4C-alkylamino is preferred and here, in particular, dimethyl-, diethyl- or diisopropylamino.

Halogen within the meaning of the invention is bromine, chlorine or fluorine.

1-4C-Alkoxycarbonyl represents a radical which, in addition to the carbonyl group, contains one of the abovementioned 1-4C-alkoxy radicals. Examples which may be mentioned are the methoxycarbonyl, the ethoxycarbonyl and the isopropoxycarbonyl radicals.

1-4C-Alkylthio represents radicals which, in addition to the sulfur atom, contain one of the abovementioned 1-4C-alkyl radicals. Examples which may be mentioned are the butylthio, propylthio and preferably the ethylthio and methylthio radicals.

Pyridyl or pyridinyl includes 2-pyridyl, 3-pyridyl and 4-pyridyl.

Mono- or di-1-4C-alkylaminocarbonyl radicals represent a radical, which in addition to the carbonyl group, contains one of the abovementioned one or two of the abovementioned mono- or di-1-4C-alkylamino radicals. Examples include, but are not restricted thereto, dimethylaminocarbonyl or diethylaminocarbonyl.

When A has the meaning “bond”, then the moiety —N(R721)R722 is directly attached to the Har1 radical.

Het1 is optionally substituted by R71, and is a monocylic 3- to 7-membered fully saturated heterocyclic ring radical,
which is bonded via a ring carbon atom to the carbonyl moiety of the carbonylamino group C(O)N(R61)-, and
which comprises one nitrogen atom and optionally one further heteroatom selected from the group consisting of nitrogen, oxygen and sulfur, and, optionally,
to which ring one or two oxo groups are bonded.

More precisely, Het1 is optionally substituted by R71 on a ring nitrogen or ring carbon atom.

The term “oxo substituent” as used herein refers to a doubly carbon-bonded oxygen atom, which form together with the carbon atom to which it is attached a carbonyl or keto group (C═O). An oxo group which is a substituent of a saturated (hetero)cyclic ring results in a conversion of —CH₂—to —C(═O)— at its binding position.

Het1 may include, without being restricted thereto, aziridinyl, azetidinyl, pyrrolidinyl, piperidinyl, homopiperidinyl, morpholinyl, thiomorpholinyl, oxazolidinyl, isoxazolidinyl, thiazolidinyl, isothiazolidinyl, pyrazolidinyl, imidazolidinyl, piperazinyl or homopiperazinyl; or 1,4-diazepan-5-onyl, piperidin-2-onyl, piperidin-4-onyl, piperazin-2-onyl, pyrrolidin-2-onyl, imidazolidin-2-onyl, glutarimidyl or succinimidyl.

In detailed example, Het1 may include, without being restricted thereto, piperidin-3-yl, morpholin-3-yl or piperidin-4-yl; or pyrrolidin-2-on-5-yl.

As further examples for Het1 according to this invention may be mentioned, without being restricted thereto, R71-substituted derivatives of the abovementioned exemplary Het1, notably, for example, Het1 radicals, which are substituted by R71 on a ring nitrogen atom.

Illustratively, as exemplary suitable Het1 radical may be mentioned, for example, without being restricted thereto, piperidin-4-yl or pyrrolidin-2-on-5-yl.

Har1 is optionally substituted by R72 and/or R73, and is a 5- or 6-membered monocyclic unsaturated (heteroaromatic) heteroaryl radical comprising 1 to 4 heteroatoms selected independently from the group consisting of oxygen, nitrogen and sulfur.

More precisely, in one embodiment according to this invention, Har1 is optionally substituted by R72 and/or R73, and is a 6-membered monocyclic unsaturated (heteroaromatic) heteroaryl radical comprising 1 to 3, particularly 1 or 2, nitrogen atoms.

In addition, in another embodiment, Har1 is optionally substituted by R72 and/or R73, and is a 5-membered monocyclic unsaturated (heteroaromatic) heteroaryl radical comprising 1 to 4 heteroatoms selected independently from the group consisting of oxygen, nitrogen and sulfur.

Preferably, the radical Har1 is bonded via a ring carbon atom to the carbonyl moiety of the C(O)N(R61) group.

Har1 may include, without being restricted thereto, furanyl, thiophenyl, pyrrolyl, oxazolyl, isoxazolyl, thiazolyl, isothiazolyl, imidazolyl, pyrazolyl, triazolyl (precisely: 1,2,4-triazolyl or 1,2,3-triazolyl), thiadiazolyl (precisely: 1,3,4-thiadiazolyl, 1,2,5-thiadiazolyl, 1,2,3-thiadiazolyl or 1,2,4-thiadiazolyl), oxadiazolyl (precisely: 1,3,4-oxadiazolyl, 1,2,5-oxadiazolyl, 1,2,3-oxadiazolyl or 1,2,4-oxadiazolyl) or tetrazolyl, or, pyridinyl, pyrimidinyl, pyrazinyl or pyridazinyl, as well as the R72- and/or R73-substituted derivatives thereof.

In more detailed example, Har1 radicals may include, without being restricted thereto, pyridinyl, pyrimidinyl, pyrazinyl or pyridazinyl, as well as the R72- and/or R73-substituted derivatives thereof.

In still more detailed example, Har1 radicals may include, without being restricted thereto, pyrimidinyl, pyrazinyl, pyridazinyl or, in particular, pyridinyl, as well as the R72- and/or R73-substituted derivatives of these radicals, wherein
R72 is 1-4C-alkoxy,
R73 is 1-4C-alkoxy.

Illustratively, as exemplary suitable Har1 radical may be mentioned, for example, without being restricted thereto, pyridinyl, or, more specifically, pyridin-4-yl or pyridin-3-yl, or the dimethoxy-substituted derivatives thereof.

As more specific exemplary suitable Har1 radical may be mentioned, for example, without being restricted thereto, 2,6-dimethoxy-pyridin-3-yl.

Het2 is optionally substituted by R723 and stands for a monocylic 3- to 7-membered fully saturated or unsaturated (heteroaromatic) heterocyclic ring radical comprising the nitrogen atom, to which R721 and R722 are bonded, and optionally one to three further heteroatoms independently selected from the group consisting of nitrogen, oxygen and sulfur.

In a first facet (facet 1) according to this invention, Het2 is optionally substituted by R723 on a ring nitrogen atom and stands for a monocylic 3- to 7-membered fully saturated heterocyclic ring radical comprising the nitrogen atom, to which R721 and R722 are bonded, and optionally one further heteroatom selected from the group consisting of nitrogen, oxygen and sulfur.

In a second facet (facet 2) according to this invention, Het2 stands for a monocylic 5-membered unsaturated (heteroaromatic) ring radical comprising the nitrogen atom, to which R721 and R722 are bonded, and optionally one to three further nitrogen atoms.

Het2 may include according to facet 1, without being restricted thereto, aziridinyl, azetidinyl, pyrrolidinyl, piperidinyl, homopiperidinyl, morpholinyl, thiomorpholinyl, oxazolidinyl, isoxazolidinyl, thiazolidinyl, isothiazolidinyl, pyrazolidinyl, imidazolidinyl, piperazinyl or homopiperazinyl.

Het2 may also include according to facet 2, without being restricted thereto, pyrrolyl, imidazolyl, pyrazolyl, triazolyl or tetrazolyl.

As further examples for Het2 according to this invention may be mentioned, without being restricted thereto, R723-substituted derivatives of the abovementioned exemplary Het2 radicals according to facet 1, such as e.g. 4-N—(R723)-piperazinyl or 4-N—(R723)-homopiperazinyl.

Illustratively, as an exemplary suitable Het2 radical may be mentioned, for example, without being restricted thereto, morpholin-4-yl.

Het3 is optionally substituted by R811, and is a 3- to 7-membered saturated monocyclic heterocyclic ring radical comprising the nitrogen atom, to which R81 and R82 are bonded, and optionally one further heteroatom selected from the group consisting of oxygen, nitrogen and sulfur.

Het3 may include, without being restricted thereto, aziridinyl, azetidinyl, pyrrolidinyl, piperidinyl, homopiperidinyl, morpholinyl, thiomorpholinyl, oxazolidinyl, isoxazolidinyl, thiazolidinyl, isothiazolidinyl, pyrazolidinyl, imidazolidinyl, piperazinyl or homopiperazinyl.

As further examples for Het3 according to this invention may be mentioned, without being restricted thereto, R811-substituted derivatives of the abovementioned exemplary Het3 radicals, notably, for example, Het3 radicals, which are substituted by R811 on a ring nitrogen atom, such as e.g. 4-N—(R811)-piperazinyl or 4-N—(R811)-homopiperazinyl.

Illustratively, as an exemplary suitable Het3 radical may be mentioned, for example, without being restricted thereto, piperidin-1-yl.

In general, unless otherwise noted, the heterocyclic groups mentioned herein refer to all of the possible isomeric forms thereof.

The heterocyclic groups mentioned herein refer, unless otherwise noted, in particular to all of the possible positional isomers thereof.

Thus, for example, the term pyridyl or pyridinyl includes pyridin-2-yl, pyridin-3-yl and pyridin-4-yl.

Constituents which are optionally substituted as stated herein, may be substituted, unless otherwise noted, at any possible position.

The heterocyclic groups, alone or as part of other groups, mentioned herein may be substituted by their given substituents, unless otherwise noted, at any possible position, such as e.g. at any substitutable ring carbon or ring nitrogen atom.

Unless otherwise noted, rings containing quaternizable imino-type ring nitrogen atoms (—N═) may be preferably not quaternized on these imino-type ring nitrogen atoms by the mentioned substituents.

Unless otherwise noted, any heteroatom of a heterocyclic ring with unsatisfied valences mentioned herein is assumed to have the hydrogen atom(s) to satisfy the valences.

When any variable occurs more than one time in any constituent, each definition is independent.

As it is known for the person skilled in the art, compounds comprising nitrogen atoms can form N-oxides. Particularly, imine nitrogen, especially heterocyclic or heteroaromatic imine nitrogen, or pyridine-type nitrogen (═N—) atoms, can be N-oxidized to form the N-oxides comprising the group ═N⁺(O⁻)—. Thus, the compounds according to the present invention comprising the imine nitrogen atom in position 5 of the phenylphenanthridine backbone and, optionally (depending on the meaning of R7), one or more further nitrogen atoms suitable to exist in the N-oxide state (═N⁺(O⁻)—) may be capable to form (depending on the number of nitrogen atoms suitable to form stabile N-oxides) mono-N-oxides, bis-N-oxides or multi-N-oxides, or mixtures thereof.

The term N-oxide(s) as used in this invention therefore encompasses all possible, and in particular all stabile, N-oxide forms, such as mono-N-oxides, bis-N-oxides or multi-N-oxides, or mixtures thereof in any mixing ratio.

Possible salts for compounds of the formula I —depending on substitution—are all acid addition salts or all salts with bases. Particular mention may be made of the pharmacologically tolerable salts of the inorganic and organic acids and bases customarily used in pharmacy. Those suitable are, on the one hand, water-insoluble and, particularly, water-soluble acid addition salts with acids such as, for example, hydrochloric acid, hydrobromic acid, phosphoric acid, nitric acid, sulfuric acid, acetic acid, citric acid, D-gluconic acid, benzoic acid, 2-(4-hydroxybenzoyl)benzoic acid, butyric acid, sulfosalicylic acid, maleic acid, lauric acid, malic acid, fumaric acid, succinic acid, oxalic acid, tartaric acid, embonic acid, stearic acid, toluenesulfonic acid, methanesulfonic acid or 3-hydroxy-2-naphthoic acid, it being possible to employ the acids in salt preparation—depending on whether a mono- or polybasic acid is concerned and depending on which salt is desired—in an equimolar quantitative ratio or one differing therefrom.

On the other hand, salts with bases are also suitable. Examples of salts with bases which may be mentioned are alkali metal (lithium, sodium, potassium) or calcium, aluminum, magnesium, titanium, ammonium, meglumine or guanidinium salts, where here too the bases are employed in salt preparation in an equimolar quantitative ratio or one differing therefrom.

Pharmacologically intolerable salts which can initially be obtained, for example, as process products in the preparation of the compounds according to the invention on an industrial scale are converted into pharmacologically tolerable salts by processes known to the person, skilled in the art.

It is known to the person skilled in the art that the compounds according to the invention and their salts, when they are isolated, for example, in crystalline form, can contain various amounts of solvents. The invention therefore also comprises all solvates and in particular all hydrates of the compounds of the formula I, and also all solvates and in particular all hydrates of the salts of the compounds of the formula I.

The substituents R6 and R7C(O)N(R61)- of compounds of formula I can be attached in the ortho, meta or para position with respect to the binding position in which the 6-phenyl ring is bonded to the phenanthridine ring system, whereby preference is given to the attachement of R7C(O)N(R61)- in the meta or in the para position. In a particular embodiment R6 is hydrogen, and the radical R7C(O)N(R61)- is attached in the meta or in the para position with respect to the binding position in which the 6-phenyl ring is bonded to the phenanthridine ring system.

Compounds of formula I to be more worthy to be mentioned are those in which
R1 is 1-2C-alkoxy, 3-5C-cycloalkoxy, 3-5C-cycloalkylmethoxy, 2,2-difluoroethoxy, or completely or predominantly fluorine-substituted 1-2C-alkoxy,
R2 is 1-2C-alkoxy, 3-5C-cycloalkoxy, 3-5C-cycloalkylmethoxy, 2,2-difluoroethoxy, or completely or predominantly fluorine-substituted 1-2C-alkoxy,
R3 is hydrogen,
R31 is hydrogen,
   either, in a first embodiment (embodiment a) according to the present invention,
R4 is —O—R41, in which
R41 is hydrogen or 1-4C-alkylcarbonyl, and
R5 is hydrogen,
   or, in a second embodiment (embodiment b) according to the present invention,
R4 is hydrogen, and
R5 is —O—R51, in which
R51 is hydrogen or 1-4C-alkylcarbonyl,
R6 is hydrogen,
R61 is hydrogen,
R7 is Het1, Har1, 3-7C-cycloalkyl, or 1-4C-alkyl substituted by R8, in which
Het1 is optionally substituted by R71 and is a monocylic 5- to 7-membered fully saturated heterocyclic ring radical,
which is bonded via a ring carbon atom to the carbonyl moiety of the —C(O)N(R61)- group, and
which comprises one nitrogen atom and optionally one further heteroatom selected from the group consisting of nitrogen, oxygen and sulfur, and, optionally,
to which ring one or two oxo groups are bonded, in which
R71 is 1-4C-alkyl, or completely or partially fluorine-substituted 1-4C-alkyl,
Har1 is optionally substituted by R72 and/or R73, and is
either a 6-membered monocyclic unsaturated heteroaryl radical comprising one or two nitrogen atoms,
or a 5-membered monocyclic unsaturated heteroaryl radical comprising 1 to 4 heteroatoms selected independently from the group consisting of oxygen, nitrogen and sulfur, in which
R72 is halogen, 1-4C-alkyl, 1-4C-alkoxy, 1-4C-alkoxy-2-4C-alkoxy, 1-4C-alkylthio, cyano, 1-4C-alkoxycarbonyl, carboxyl, hydroxyl, -A-N(R721)R722, pyridyl, or completely or partially fluorine-substituted 1-4C-alkyl, in which
A is a bond or 1-4C-alkylene,
R721 is hydrogen or 1-4C-alkyl,
R722 is hydrogen or 1-4C-alkyl,
or R721 and R722 together and with inclusion of the nitrogen atom, to which they are attached, form a heterocyclic ring Het2, in which
Het2 is optionally substituted by R723, and is a 3- to 7-membered saturated or unsaturated monocyclic heterocyclic ring radical comprising the nitrogen atom, to which R721 and R722 are bonded, and optionally one to three further heteroatoms independently selected from the group consisting of oxygen, nitrogen and sulfur, in which
R723 is 1-4C-alkyl,
R73 is halogen; 1-4C-alkoxy, 1-4C-alkoxy-2-4C-alkoxy, 1-4C-alkylthio, hydroxyl, amino or mono- or di-1-4C-alkylamino,
R8 is 1-4C-alkoxy, carbamoyl, mono- or di-1-4C-alkylaminocarbonyl or —N(R81)R82, in which
R81 is hydrogen, 1-4C-alkyl, carbamoyl, amidino or 1-4C-alkylcarbonyl,
R82 is hydrogen or 1-4C-alkyl,
or R81 and R82 together and with inclusion of the nitrogen atom, to which they are attached, form a heterocyclic ring Het3, in which
Het3 is optionally substituted by R811, and is a 3- to 7-membered saturated monocyclic heterocyclic ring radical comprising the nitrogen atom, to which R81 and R82 are bonded, and optionally one further heteroatom selected from the group consisting of oxygen, nitrogen and sulfur, in which
R811 is 1-4C-alkyl,
   and the salts, the N-oxides and the salts of the N-oxides of these compounds.

Compounds of formula I in particular worthy to be mentioned are those in which R1 is 1-2C-alkoxy, 3-5C-cycloalkoxy, 3-5C-cycloalkylmethoxy, 2,2-difluoroethoxy, or completely or predominantly fluorine-substituted 1-2C-alkoxy,
R2 is 1-2C-alkoxy, 3-5C-cycloalkoxy, 3-5C-cycloalkylmethoxy, 2,2-difluoroethoxy, or completely or predominantly fluorine-substituted 1-2C-alkoxy,
R3 is hydrogen,
R31 is hydrogen,
R4 is —O—R41, in which
R41 is 1-4C-alkylcarbonyl or, in particular, in an individual embodiment according to this invention, hydrogen,
R5 is hydrogen,
R6 is hydrogen,
R61 is hydrogen,
R7 is Het1, Har1, 3-7C-cycloalkyl, or 1-4C-alkyl substituted by R8, in which
Het1 is optionally substituted by R71 and is a monocylic 5- to 7-membered fully saturated heterocyclic ring radical,
which is bonded via a ring carbon atom to the carbonyl moiety of the —C(O)N(R61)- group, and
which comprises one nitrogen atom, and, optionally,
to which ring one oxo group is bonded, in which
R71 is 1-4C-alkyl, or completely or partially fluorine-substituted 1-2C-alkyl,
Har1 is optionally substituted by R72 and/or R73, and is
a 6-membered monocyclic unsaturated heteroaryl radical comprising one or two nitrogen atoms, in which
R72 is halogen, 1-4C-alkoxy, 1-4C-alkoxy-ethoxy, 1-2C-alkylthio, hydroxyl, amino or mono- or di-1-2C-alkylamino,
R73 is halogen, 1-4C-alkoxy, 1-2C-alkoxy-ethoxy, 1-2C-alkylthio, hydroxyl, amino or mono- or di-1-2C-alkylamino,
R8 is 1-4C-alkoxy, carbamoyl, mono- or di-1-4C-alkylaminocarbonyl or —N(R81)R82, in which
R81 is hydrogen, 1-4C-alkyl, carbamoyl or 1-4C-alkylcarbonyl,
R82 is hydrogen or 1-4C-alkyl,
or R81 and R82 together and with inclusion of the nitrogen atom, to which they are attached, form a heterocyclic ring Het3, in which
Het3 is optionally substituted by R811, and is a 3- to 7-membered saturated monocyclic heterocyclic ring radical comprising the nitrogen atom, to which R81 and R82 are bonded, and optionally one further heteroatom selected from the group consisting of oxygen, nitrogen and sulfur, in which
R811 is 1-2C-alkyl,
   and the salts, the N-oxides and the salts of the N-oxides of these compounds.

Compounds of formula I in more particular worthy to be mentioned are those in which
R1 is 1-2C-alkoxy, 2,2-difluoroethoxy, or completely or predominantly fluorine-substituted 1-2C-alkoxy,
R2 is 1-2C-alkoxy, 2,2-difluoroethoxy, or completely or predominantly fluorine-substituted 1-2C-alkoxy,
R3 is hydrogen,
R31 is hydrogen,
R4 is —O—R41, in which
R41 is hydrogen,
R5 is hydrogen,
R6 is hydrogen,
R61 is hydrogen,
R7 is Het1, Har1, 3-5C-cycloalkyl, or 1-4C-alkyl substituted by R8, in which
Het1 is 1-N—(R71)-piperdin-4-yl or pyrrolidin-2-on-5-yl, in which
R71 is 1-4C-alkyl,
Har1 is optionally substituted by R72 and/or R73, and is pyridinyl, in which
R72 is 1-4C-alkoxy,
R73 is 1-4C-alkoxy,
R8 is 1-4C-alkoxy, carbamoyl, mono- or di-1-4C-alkylaminocarbonyl or —N(R81)R82, in which
R81 is 1-4C-alkyl, carbamoyl or 1-4C-alkylcarbonyl,
R82 is hydrogen or 1-4C-alkyl,
or R81 and R82 together and with inclusion of the nitrogen atom, to which they are attached, form a heterocyclic ring Het3, in which
Het3 is piperidin-1-yl, pyrrolidin-1-yl, 4-N—(R811)-piperazin-1-yl, 4-N—(R811)-homopiperazin-1-yl, homopiperidin-1-yl, morpholin-4-yl or thiomorpholin-4-yl, in which
R811 is 1-2C-alkyl,
   and the salts, the N-oxides and the salts of the N-oxides of these compounds.

Compounds of formula I in still more particular worthy to be mentioned are those in which
R1 is 1-2C-alkoxy, 2,2-difluoroethoxy, or completely or predominantly fluorine-substituted 1-2C-alkoxy,
R2 is 1-2C-alkoxy, 2,2-difluoroethoxy, or completely or predominantly fluorine-substituted 1-2C-alkoxy,
R3 is hydrogen,
R31 is hydrogen,
R4 is —O—R41, in which
R41 is hydrogen,
R5 is hydrogen,
R6 is hydrogen,
R61 is hydrogen,
R7 is Het1, Har1, 3-5C-cycloalkyl, or 1-4C-alkyl substituted by R8, in which
Het1 is 1-N—(R71)-piperdin-4-yl or pyrrolidin-2-on-5-yl, in which
R71 is 1-4C-alkyl,
Har1 is 2,6-dimethoxypyridin-3-yl,
R8 is 1-4C-alkoxy, carbamoyl, mono- or di-1-4C-alkylaminocarbonyl or —N(R81)R82, in which
R81 is 1-4C-alkyl, carbamoyl or 1-4C-alkylcarbonyl,
R82 is hydrogen or 1-4C-alkyl,
or R81 and R82 together and with inclusion of the nitrogen atom, to which they are attached, form a heterocyclic ring Het3, in which
Het3 is piperidin-1-yl,
   and the salts, the N-oxides and the salts of the N-oxides of these compounds.

Compounds of formula I in yet still more particular worthy to be mentioned are those in which
R1 is methoxy or ethoxy,
R2 is methoxy, ethoxy, difluoromethoxy or 2,2-difluoroethoxy,
R3 is hydrogen,
R31 is hydrogen,
R4 is —O—R41, in which
R41 is hydrogen,
R5 is hydrogen,
R6 is hydrogen,
R61 is hydrogen,
R7 is Het1, Har1, cyclopropyl, or 1-4C-alkyl substituted by R8, in which
Het1 is 1-N—(R71)-piperdin-4-yl or pyrrolidin-2-on-5-yl, in which
R71 is methyl,
Har1 is 2,6-dimethoxypyridin-3-yl,
R8 is methoxy, carbamoyl, dimethylaminocarbonyl or —N(R81)R82, in which
R81 is methyl, carbamoyl or acetyl,
R82 is hydrogen or methyl,
or R81 and R82 together and with inclusion of the nitrogen atom, to which they are attached, form a heterocyclic ring Het3, in which
Het3 is piperidin-1-yl,
   and the salts, the N-oxides and the salts of the N-oxides of these compounds.

A special interest in the compounds according to this invention relates to those compounds which are included by one or, when possible, by more of the following embodiments:

A special embodiment of the compounds of the present invention include those compounds of formula I in which R1 and R2 are independently 1-2C-alkoxy; 2,2-difluoroethoxy, or completely or predominantly fluorine-substituted 1-2C-alkoxy.

Another special embodiment of the compounds of the present invention include those compounds of formula I in which R1 and R2 are independently 1-2C-alkoxy, 2,2-difluoroethoxy, or completely or predominantly fluorine-substituted 1-2C-alkoxy, and R3 and R31 are both hydrogen.

Another special embodiment of the compounds of the present invention include those compounds of formula I in which R1 and R2 are independently 1-2C-alkoxy, 2,2-difluoroethoxy, or completely or predominantly fluorine-substituted 1-2C-alkoxy, and R3, R31 and R6 are all hydrogen.

Another special embodiment of the compounds of the present invention include those compounds of formula I in which R1 or R2 is 2,2-difluoroethoxy, and R3, R31 and R6 are all hydrogen.

Another special embodiment of the compounds of the present invention include those compounds of formula I in which one of R1 and R2 is methoxy, and the other is methoxy, ethoxy, difluoromethoxy or 2,2-difluoroethoxy, and R3 and R31 are both hydrogen.

Another special embodiment of the compounds of the present invention include those compounds of formula I in which R1 is ethoxy or, particularly, methoxy, and R2 is methoxy, or, particularly, ethoxy, difluoromethoxy or 2,2-difluoroethoxy, and R3 and R31 are both hydrogen.

Another special embodiment of the compounds of the present invention include those compounds of formula I in which R1 is methoxy, and R2 is methoxy, ethoxy, difluoromethoxy or 2,2-difluoroethoxy, and R3 and R31 are both hydrogen.

Another special embodiment of the compounds of the present invention include those compounds of formula I in which R1 is methoxy, and R2 is ethoxy, difluoromethoxy or 2,2-difluoroethoxy, and R3 and R31 are both hydrogen.

Another special embodiment of the compounds of the present invention include those compounds of formula I in which one of R1 and R2 is 2,2-difluoroethoxy, and R3 and R31 are both hydrogen.

Another special embodiment of the compounds of the present invention include those compounds of formula I in which R1 is ethoxy or, particularly, methoxy, and R2 is 2,2-difluoroethoxy, and R3 and R31 are both hydrogen.

Another special embodiment of the compounds of the present invention include those compounds of formula I in which R1 is methoxy, and R2 is 2,2-difluoroethoxy, and R3 and R31 are both hydrogen.

Another special embodiment of the compounds of the present invention include those compounds of formula I in which R1 is methoxy, and R2 is ethoxy, and R3 and R31 are both hydrogen.

Another special embodiment of the compounds of the present invention include those compounds of formula I in which R1 is methoxy, and R2 is difluoromethoxy, and R3 and R31 are both hydrogen.

Another special embodiment of the compounds of the present invention include those compounds of formula I in which R6 is hydrogen.

Another special embodiment of the compounds of the present invention include those compounds of formula I in which R61 is hydrogen.

Another special embodiment of the compounds of the present invention include those compounds of formula I, in which R5 or, particularly, R4 is the radical (1-4C-alkylcarbonyl)—O—such as e.g. acetoxy, or hydroxyl, and all the other substituents are as defined in any compound which is said to be mentioned above.

Another special embodiment of the compounds of the present invention include those compounds of formula I in which R5 or, particularly, R4 is hydroxyl.

A preferred embodiment according to the present invention is embodiment a.

A further preferred embodiment of the compounds of the present invention include compounds according to embodiment a, in which R5 and R41 are both hydrogen, and in which R1 and R2 are independently 1-2C-alkoxy, 2,2-difluoroethoxy, or completely or predominantly fluorine-substituted 1-2C-alkoxy, and R3, R31 and R6 are all hydrogen.

A yet further preferred embodiment of the compounds of the present invention include compounds according to embodiment a, in which R5 is hydrogen, and in which R1 is methoxy, and R2 is ethoxy, difluoromethoxy or 2,2-difluoroethoxy, and R3, R31 and R6 are all hydrogen.

A still yet further preferred embodiment of the compounds of the present invention include compounds according to embodiment a, in which R5 and R41 are both hydrogen, and in which R-1 is methoxy, and R2 is ethoxy, difluoromethoxy or 2,2-difluoroethoxy, and R3, R31 and R6 are all hydrogen.

Suitable compounds according to the present invention more worthy to be mentioned include those compounds of formula I, in which R5 or, particularly, R4 is hydroxyl.

Exemplary compounds according to the present invention may include those selected from N-[3-((2RS,4aRS,10bRS)-9-Ethoxy-2-hydroxy-8-methoxy-1,2,3,4,4a,10b-hexahydro-phenanthridin-6-yl)-phenyl]-2-methoxy-acetamide N-[4-((2RS,4aRS,10bRS)-9-Ethoxy-2-hydroxy-8-methoxy-1,2,3,4,4a,10b-hexahydro-phenanthridin-6-yl)-phenyl]-3-methoxy-propionamide Cyclopropanecarboxylic acid [3-((2RS,4aRS, 10bRS)-9-ethoxy-2-hydroxy-8-methoxy-1,2,3,4,4a,10b-hexahydro-phenanthridin-6-yl)-phenyl]-amide N-[4-((2RS,4aRS,10bRS)-9-Ethoxy-2-hydroxy-8-methoxy-1,2,3,4,4a,10b-hexahydro-phenanthridin-6-yl)-phenyl]-3-piperidin-1-yl-propionamide N-[4-((2RS,4aRS,10bRS)-9-Ethoxy-2-hydroxy-8-methoxy-1,2,3,4,4a,10b-hexahydro-phenanthridin-6-yl)-phenyl]-N′,N′-dimethyl-succinamide 1-Methyl-piperidine-4-carboxylic acid [4-((2RS,4aRS,10bRS)-9-ethoxy-2-hydroxy-8-methoxy-1,2,3,4,4a,10b-hexahydro-phenanthridin-6-yl)-phenyl]-amide Cyclopropanecarboxylic acid [4((2RS,4aRS,10bRS)-9-ethoxy-2-hydroxy-8-methoxy-1,2,3,4,4a, 10b-hexahydro-phenanthridin-6-yl)-phenyl]-amide N-[3-((2RS,4aRS,10bRS)-9-Ethoxy-2-hydroxy-8-methoxy-1,2,3,4,4a,10b-hexahydro-phenanthridin-6-yl)-phenyl]-3-methoxy-propionamide N-[4-((2RS,4aRS,10bRS)-9-Ethoxy-2-hydroxy-8-methoxy-1,2,3,4,4a,10b-hexahydro-phenanthridin-6-yl)-phenyl]-2-methoxy-acetamide N-[3-((2RS,4aRS,10bRS)-9-Ethoxy-2-hydroxy-8-methoxy-1,2,3,4,4a,10b-hexahydro-phenanthridin-6-yl)-phenyl]-3-piperidin-1-yl-propionamide 1-Methyl-piperidine-4-carboxylic acid [3-((2RS,4aRS,10bRS)-9-ethoxy-2-hydroxy-8-methoxy-1,2,3,4,4a,10b-hexahydro-phenanthridin-6-yl)-phenyl]-amide N-[3-((2RS,4aRS,10bRS)-9-Ethoxy-2-hydroxy-8-methoxy-1,2,3,4,4a, 10b-hexahydro-phenanthridin-6-yl)-phenyl]-N′,N′-dimethyl-succinamide Dimethylamino-N-[4-((2RS,4aRS,10bRS)-9-ethoxy-2-hydroxy-8-methoxy-1,2,3,4,4a,10b-hexahydro-phenanthridin-6-yl)-phenyl]-butyramide Dimethylamino-N-[3-((2RS,4aRS,10bRS)-9-ethoxy-2-hydroxy-8-methoxy-1,2,3,4,4a,10b-hexahydro-phenanthridin-6-yl)-phenyl]-butyramide 2-Acetylamino-N-[3-((2RS,4aRS,10bRS)-9-ethoxy-2-hydroxy-8-methoxy-1,2,3,4,4a,10b-hexahydro-phenanthridin-6-yl)-phenyl]-acetamide 5Oxo-pyrrolidine-2-carboxylic acid [3-((2RS,4aRS,10bRS)-9-ethoxy-2-hydroxy8-methoxy-1,2,3,4,4a,10b-hexahydro-phenanthridin-6-yl)-phenyl]-amide N-[3-((2RS,4aRS,10bRS)-9-Ethoxy-2-hydroxy-8-methoxy-1,2,3,4,4a,10b-hexahydro-phenanthridin-6-yl) -phenyl]-2,6-dimethoxy-nicotinamide (2RS,4aRS,10bRS)-6-[3-(3-carbamoyl-propanoylamino)-phenyl]-9-ethoxy-8-methoxy-1,2,3,4,4a,10b-hexahydro-phenanthridin-2-ol N-[3-((2RS,4aRS,10bRS)-9-Ethoxy-2-hydroxy-8-methoxy-1,2,3,4,4a,10b-hexahydro-phenanthridin-6-yl)-phenyl]-3-ureido-propionamide and Cyclopropanecarboxylic acid [3-((3SR,4aRS,10bRS)-9-ethoxy-3-hydroxy-8-methoxy-1,2,3,4,4a,10b-hexahydro-phenanthridin-6-yl)-phenyl]-amide the enantiomers, as well as the salts, the N-oxides and the salts of the N-oxides of these compounds and enantiomers.

Preferably, the compounds according to the present invention which are listed in the Table A in the appended “Biological Investigations” and, particularly, the enantiomers thereof, particularly those having the formula Ia*****, as well as the salts of these compounds and enantiomers, are to be mentioned as a particular interesting aspect of the present invention.

The compounds of formula I are chiral compounds having chiral centers at least in positions 4a and 10b and depending on the meanings of R3, R31, R4 and R5 additional chiral centers in positions 1, 2, 3 and 4.

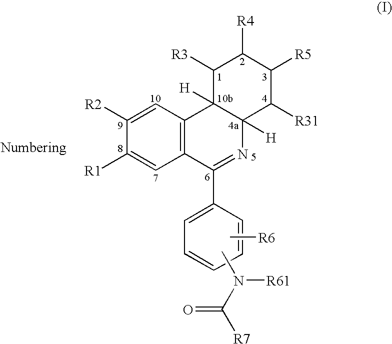

The invention includes all conceivable stereoisomers in pure form as well as in any mixing ratio. Preference is given to compounds of formula I in which the hydrogen atoms in positions 4a and 10b are in the cis position relative to one another. The pure cis enantiomers and their mixtures in any mixing ratio and including the racemates are more preferred in this context.

Particularly preferred in this context are those compounds of formula 1, which have with respect to the positions 4a and 10b the configuration shown in formula (I*):

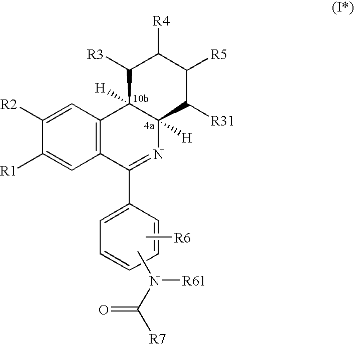

If, for example, in compounds of formula I* R3, R31 and R5 have the meaning hydrogen and R4 has the meaning —OR41, then the configuration—according to the rules of Cahn, Ingold and Prelog—is R in the 4a position and R in the 10b position.

Further preferred compounds of the formula I according to embodiment a are those which have, with respect to the positions 2, 4a and 10b, the same configuration as shown in the formulae Ia** and Ia*** and Ia****:

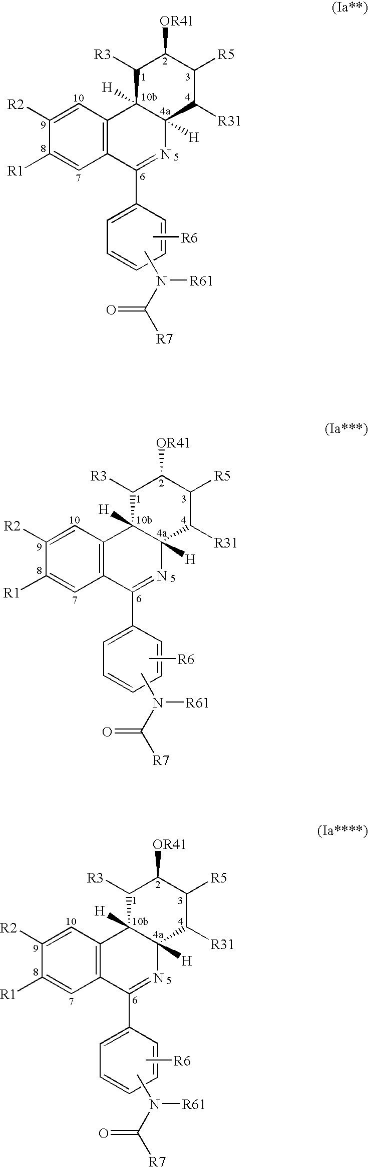

If, for example in compounds of the formula Ia** R3, R31 and R5 have the meaning hydrogen, then the configuration—according the rules of Cahn, Ingold and Prelog—is S in the position 2, R in the position 4a and R in the position 10b.

If, for example in compounds of the formula Ia*** R3, R31 and R5 have the meaning hydrogen, then the configuration—according the rules of Cahn, Ingold and Prelog—is R in the position 2, S in the position 4a and S in the position 10b.

If, for example in compounds of the formula Ia**** R3, R31 and R5 have the meaning hydrogen, then the configuration—according the rules of Cahn, Ingold and Prelog—is S in the position 2, S in the position 4a and S in the position 10b.

In more particular preferred compounds of the formula I according embodiment a are those which have, with respect to the positions 2, 4a and 10b, the same configuration as shown in the formula Ia*****:

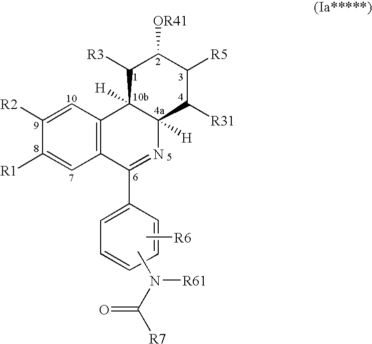

If, for example in compounds of the formula Ia***** R3, R31 and R5 have the meaning hydrogen, then the configuration—according the rules of Cahn, Ingold and Prelog—is R in the position 2, R in the position 4a and R in the position 10b.

Preferred compounds of the formula I according to embodiment b are those which have, with respect to the positions 3, 4a and 10b, the same configuration as shown in the formulae Ib** and Ib*** and Ib****:

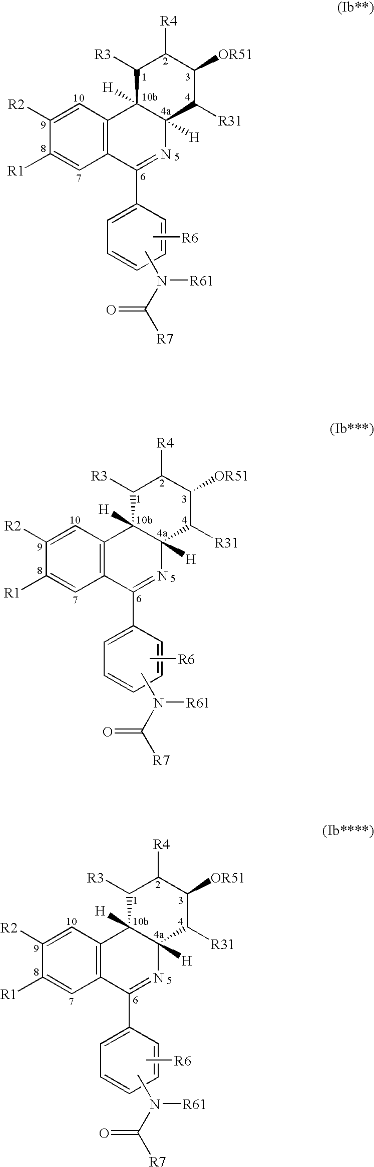

If, for example in compounds of the formula Ib** R3, R31 and R5 have the meaning hydrogen, then the configuration—according the rules of Cahn, Ingold and Prelog—is R in the position 3, R in the position 4a and R in the position 10b.

If, for example in compounds of the formula Ib*** R3, R31 and R5 have the meaning hydrogen, then the configuration—according the rules of Cahn, Ingold and Prelog—is S in the position 3, S in the position 4a and S in the position 10b.

If, for example in compounds of the formula Ib**** R3, R31 and R5 have the meaning hydrogen, then the configuration—according the rules of Cahn, Ingold and Prelog—is R in the position 3, S in the position 4a and S in the position 10b.

More preferred compounds of the formula I according to embodiment b are those which have, with respect to the positions 3, 4a and 10b, the same configuration as shown in the formula Ib*****:

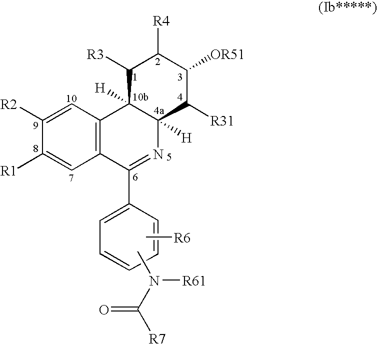

If, for example in compounds of the formula Ib***** R3, R31 and R5 have the meaning hydrogen, then the configuration—according the rules of Cahn, Ingold and Prelog—is S in the position 3, R in the position 4a and R in the position 10b.

Within the meaning of the embodiments a and b according to this invention, compounds of formula Ia***** are in particular to be emphasized.

The enantiomers can be separated in a manner known per se (for example by preparation and separation of appropriate diastereoisomeric compounds). Thus, e.g. an enantiomer separation can be carried out at the stage of the starting compounds having a free amino group such as starting compounds of formulae VIa, in which R1, R2, R3, R31, R41 and R5 have the meanings mentioned above, or IXb as defined below.

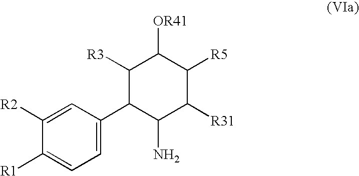

Separation of the enantiomers can be carried out, for example, by means of salt formation of the racemic compounds of the formulae VIa or IXb with optically active acids, preferably carboxylic acids, subsequent resolution of the salts and release of the desired compound from the salt. Examples of optically active carboxylic acids which may be mentioned in this connection are the enantiomeric forms of mandelic acid, tartaric acid, O,O′-dibenzoyltartaric acid, camphoric acid, quinic acid, glutamic acid, pyroglutamic acid, malic acid, camphorsulfonic acid, 3-bromocamphorsulfonic acid, α-methoxyphenylacetic acid, α-methoxy-α-trifluoromethylphenylacetic acid and 2-phenylpropionic acid. Alternatively, enantiomerically pure starting compounds can be prepared via asymmetric syntheses. Enantiomerically pure starting compounds as well as enantiomerically pure compounds of the formula I can be also obtained by chromatographic separation on chiral separating columns; by derivatization with chiral auxiliary reagents, subsequent diastereomer separation and removal of the chiral auxiliary group; or by (fractional) crystallization from a suitable solvent.

The compounds according to the invention can be prepared, for example, as shown in the reaction schemes below and according to the following specified reaction steps, or, particularly, in a manner as described by way of example in the following examples, or analogously or similarly thereto according to preparation procedures or synthesis strategies known to the person skilled in the art.

Compounds of formula I, in which R1, R2, R3, R31, R4, R5, R6, R61 and R7 have the meanings mentioned above, can be obtained as outlined in reaction scheme 1 and as described as follows starting from the compounds of formula III, in which R1, R2, R3, R31, R4, R5 and R6 have the meanings mentioned above whereby R4 and R5 are other than hydroxyl, by reduction of the nitro group of compounds of formula III to give corresponding compounds of formula II. Said reduction can be carried out in a manner known to the person skilled in the art or as described exemplarily in the following examples using, for example, a suitable reducing agent such as e.g. tin dichloride, or via a hydrogenation reaction in the presence of a suitable metal catalyst.

In the next step compounds of formula II are reacted with carboxylic acid derivatives of the formula R7-C(O)—X, in which X is a suitable leaving group, for example a chlorine atom.

Alternatively, compounds of formula I can also be prepared from compounds of the formula II, in which R1, R2, R3, R31, R4, R5 and R6 have the meanings mentioned above whereby R4 and R5 are other than hydroxyl, and compounds of formula R7-C(O)—X, in which X is hydroxyl, by reaction with amide bond linking reagents (=coupling agents) optionally in the presence of an auxiliary known to the person skilled in the art. Exemplary amide bond linking reagents known to the person skilled in the art which may be mentioned are, for example, the carbodiimides (e.g. dicyclohexylcarbodiimide or, preferably, 1-ethyl-3-(3-dimethylaminopropyl)carbodiimide hydrochloride), azodicarboxylic acid derivatives (e.g. diethyl azodicarboxylate), uronium salts [e.g. O-(benzotriazol-1-yl)-N,N,N′,N′-tetramethyluronium tetrafluoroborate or O-(benzotriazol-1yl)-N,N,N′,N′-tetramthyluronium-hexafluorophosphate] and N,N′-carbonyidiimidazole. In the scope of this invention preferred amide bond linking reagents are uronium salts and, particularly, carbodiimides, preferably, 1-ethyl-3-(3-dimethylaminopropyl)carbodiimide hydrochloride.

Optionally, the amide compounds of formula I obtained, in which R61 is hydrogen, can be N-alkylated using a suitable base to deprotonate the amide (e.g. sodium hydride) and a suitable alkylating agent R61-Y, in which Y is a suitable leaving group and R61 is other than hydrogen, to give compounds of formula I, in which R61 is other than hydrogen and has the meanings as given above.

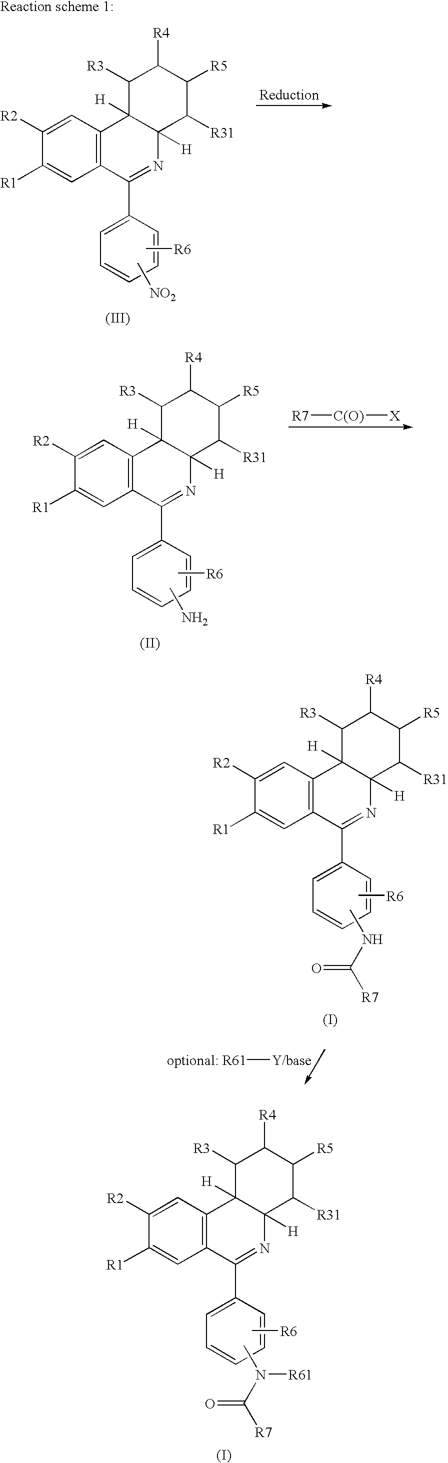

Optionally, compounds of the formula I can be also converted into further compounds of the formula I by methods known to one of ordinary skill in the art. More specifically, for example, from compounds of the formula I in which
a) R41 or R51 is hydrogen, the corresponding ester compounds can be obtained by esterification reactions;
b) R41 or R51 is hydrogen, the corresponding ether compounds can be obtained by etherification reactions;
c) R41 or R51 is an acyl group, such as e.g. acetyl, the corresponding hydroxyl compounds can be obtained by deesterification (e.g. saponification) reactions;
d) R61 is hydrogen, the corresponding N-ether compounds can be obtained by etherification reaction;

The methods mentioned under a), b), c) and d) are expediently carried out analogously to the methods known to the person skilled in the art or as described by way of example in the following examples.

Optionally, compounds of the formula I can be converted into their salts, or, optionally, salts of the compounds of the formula I can be converted into the free compounds.

In addition, the compounds of the formula I can be converted, optionally, into their N-oxides, for example with the aid of hydrogen peroxide in methanol or with the aid of m-chloroperoxybenzoic acid in dichloromethane. The person skilled in the art is familiar on the basis of his/her expert knowledge with the reaction conditions which are specifically necessary for carrying out the N-oxidation.

Compounds of formula III according to embodiment a or b (i.e. compounds of formulae III or IIIb, respectively) can be obtained as described as follows.

Compounds of formula IIa according to embodiment a, in which R1, R2, R3, R31, R41, R5 and R6 have the meanings mentioned above in embodiment a whereby R41 is other than hydrogen, can be prepared as described and shown in reaction scheme 2 below.

In the first reaction step of the synthesis route shown in scheme 2, compounds of the formula VIIa, in which R1, R2, R3, R31, R41 and R5 have the meanings mentioned above in embodiment a whereby R41 is other than hydrogen, are prepared from the corresponding compounds of the formula VIIIa by introduction of the group R41, which is other than hydrogen. The introduction reaction is carried out in a manner habitual per se for an etherification or esterification reaction, or as described by way of example in the following examples.

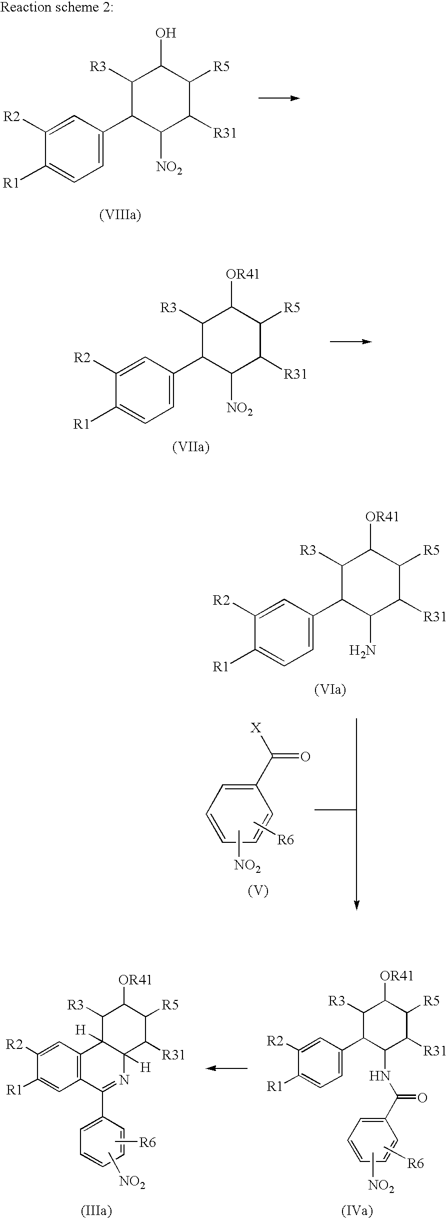

In the next reaction step of the synthesis route shown in reaction scheme 2, the nitro group of compounds of the formula VIIa, in which R1, R2, R3, R31, R41 and R5 have the meanings mentioned above in embodiment a whereby R41 is other than hydrogen, is reduced to the amino group of the corresponding compounds of the formula VIa. Said reduction is carried out in a manner known to the person skilled in the art, for example as described in J. Org. Chem. 1962, 27, 4426 or as described in the following examples. In more detail, the reduction can be carried out, for example, by catalytic hydrogenation, e.g. in the presence of Raney nickel or a noble metal catalyst such as palladium on active carbon, in a suitable solvent such as methanol or ethanol at room temperature and under normal or elevated pressure. Optionally, a catalytic amount of an acid, such as, for example, hydrochloric acid, can be added to the solvent. Preferably, however, the reduction is carried out using a hydrogen-producing mixture, for example, metals such as zinc, zinc-copper couple or iron with organic acids such as acetic acid or mineral acids such as hydrochloric acid. More preferably, the reduction is carried out using a zinc-copper couple in the presence of an organic or an inorganic acid. Such a zinc-copper couple is accessible in a way known to the person of ordinary skill in the art.

Compounds of the formula VIa, in which R1, R2, R3, R31, R41 and R5 have the meanings indicated above in embodiment a whereby R41 is other than hydrogen and which are sensitive against catalytic hydrogenation, can be prepared from the corresponding compounds of the formula VIIa by selective reduction of the nitro group in a manner known to the person skilled in the art, for example by hydrogen transfer reaction in the presence of a metal catalyst, for example palladium or, preferably, Raney nickel, in a lower alcohol as solvent using, for example, ammonium formiate or, preferably, hydrazine hydrate as hydrogen donor.

Compounds of the formula IVa, in which R1, R2, R3, R31, R41, R5 and R6 have the meanings indicated above in embodiment a whereby R41 is other than hydrogen, are accessible from the corresponding compounds of the formula VIa by reaction with corresponding compounds of the formula V, in which X represents a suitable leaving group, preferably a chlorine atom.

Alternatively, compounds of the formula IVa can also be prepared from the corresponding compounds of the formula VIa and corresponding compounds of the formula V, in which X is hydroxyl, by reaction with amide bond linking reagents known to the person skilled in the art. Exemplary amide bond linking reagents known to the person skilled in the art which may be mentioned are, for example, the carbodiimides (e.g. dicyclohexylcarbodiimide or, preferably, 1-ethyl-3-(3-dimethylaminopropyl)carbodiimide hydrochloride), azodicarboxylic acid derivatives (e.g. diethyl azodicarboxylate), uronium salts [e.g. O-(benzotriazol-1-yl)-N,N,N′,N′-tetramethyluronium tetrafluoroborate or O-(benzotriazol-1yl)-N,N,N′,N′-tetramthyl-uronium-hexafluorophosphate] and N,N′-carbonyldiimidazole. In the scope of this invention preferred amide bond linking reagents are uronium salts and, particularly, carbodiimides, preferably, 1-ethyl-3-(3-dimethylaminopropyl)carbodiimide hydrochloride.

Compounds of the formula V are either known or can be prepared in a known manner.

Compounds of the formula IIIa, in which R1, R2, R3, R31, R41, R5 and R6 have the meanings mentioned in embodiment a whereby R41 is other than hydrogen, can be obtained by cyclocondensation of corresponding compounds of the formula IVa.

Said cyclocondensation reaction is carried out in a manner known per se to the person skilled in the art or as described by way of example in the following examples, according to Bischler-Napieralski (e.g. as described in J. Chem. Soc., 1956, 4280-4282) in the presence of a suitable condensing agent, such as, for example, polyphosphoric acid, phosphorus pentachloride, phosphorus pentoxide or phosphorus oxychloride, in a suitable inert solvent, e.g. in a chlorinated hydrocarbon such as chloroform, or in a cyclic hydrocarbon such as toluene or xylene, or another inert solvent such as isopropyl acetate or acetonitrile, or without further solvent using an excess of condensing agent, at reduced temperature, or at room temperature, or at elevated temperature or at the boiling temperature of the solvent or condensing agent used. If necessary, said cyclocondensation reaction can be carried out in the presence of one or more suitable Lewis Acids such as, for example, suitable metal halogenides (e.g. chlorides) or sulphonates (e.g. triflates), including rare earth metal salts, such as e.g. anhydrous aluminum trichloride, aluminum tribromide, zinc chloride, boron trifluoride ethereate, titanium tetrachloride or, in particular, tin tetrachloride, and the like.

Below reaction scheme 3 shows the synthesis of compounds of the formula VIIIa, in which R1, R2, R3, R31 and R5 have the meanings indicated above in embodiment a, from corresponding compounds of the formula IXa via reduction reaction of the carbonyl group. Suitable reducing agents for the abovementioned reduction reaction may include, for example, metal hydride compounds such as, for example, diisopropylaluminium hydride, borane, sodium borohydride, sodium triacetoxyborohydride, sodium cyanoborohydride, zinc borohydride, potassium tri-sec-butylborohydride, sodium tri-sec-butylborohydride, lithium trisec-butylborohydride, β-isopinocampheyl-9-borabicyclo[3.3.1]nonane and the like. The preferred examples of said reducing agents are sodium cyanoborohydride, β-isopinocampheyl-9-borabicyclo[3.3.1]nonane and potassium tri-sec-butylborohydride. The most preferred examples of the abovementioned reducing agents are β-isopinocampheyl-9-borabicyclo[3.3.1]nonane and potassium trisec-butylborohydride, which both allow to prepare compounds of the formula VIa stereoselectively. “Stereoselectively” in this connection means that those compounds of the formula VIa, in which the hydrogen atoms in positions 1 and 3 are located at the opposite side of the plane defined by the cyclohexane ring, are obtained preferentially.

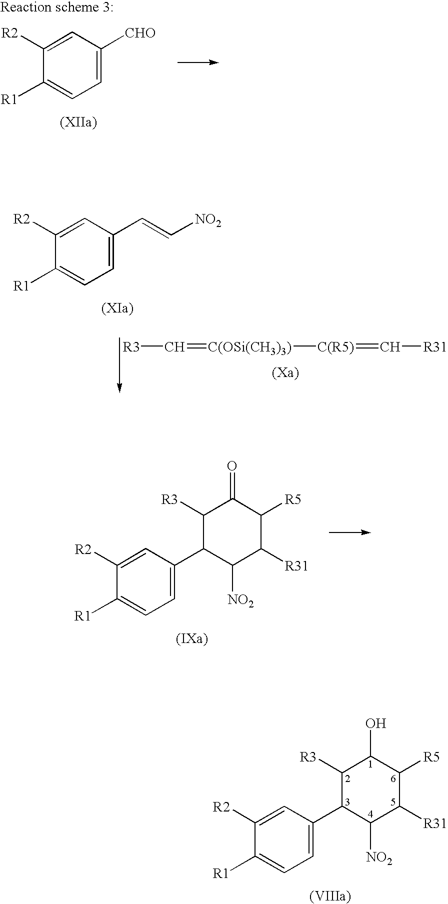

The compounds of the formula IXa, in which R1, R2, R3, R31 and R5 have the meanings mentioned in embodiment a, are either known or can be obtained by the reaction of compounds of the formula XIa, in which R1 and R2 have the meanings mentioned above, with compounds of the formula Xa, in which R3, R31 and R5 have the meanings mentioned above in embodiment a. The cycloaddition reaction is carried out in a manner known to the person skilled in the art according to Diels-Alder, e.g. as described in J. Amer. Chem. Soc. 1957, 79, 6559 or in J. Org. Chem. 1952, 17, 581 or as described in the following examples.

Compounds of the formulae VIIIa or VIIa, in which the phenyl ring and the nitro group are trans to one another, can be converted in a manner known to the person skilled in the art into the corresponding cis compounds, e.g. as described in J. Amer. Chem. Soc. 1957, 79, 6559 or as described in the following examples.

The compounds of the formulae Xa and XIa are either known or can be prepared in a known manner. The compounds of the formula XIa can be prepared, for example, in a manner known to the person skilled in the art from corresponding compounds of the formula XIIa as described, for example, in J. Chem. Soc. 1951, 2524 or in J. Org. Chem. 1944, 9, 170 or as described in the following examples.

The compounds of the formula XIIa, in which R1 and R2 have the meanings indicated above in embodiment a, are either known or can be prepared in a manner known to the person skilled in the art, as described, for example, in Ber. Dtsch. Chem. Ges. 1925, 58, 203.

Compounds of formula IIIb according to embodiment b, in which R1, R2, R3, R31, R4 and R51 have the meanings indicated above in embodiment b whereby R51 is other than hydrogen, can be prepared as described and shown in reaction scheme 4 below.

In the first reaction step in reaction scheme 4, the nitro group of compounds of the formula Xb, in which R1, R2, R3, R31 and R4 have the meanings indicated in embodiment b above, is reduced to obtain corresponding compounds of the formula IXb. Said reduction reaction is carried out in a manner known to the person skilled in the art, for example as described in J. Org. Chem. 1962, 27, 4426 or as described in the following examples. More specifically, the reduction can be carried out, for example, by contacting compounds of the formula VIIb with a hydrogen-producing mixture such as, preferably, metallic zinc in a mildly acidic medium such as acetic acid in a lower alcohol such as methanol or ethanol at room temperature or at elevated temperature or, preferably, at the boiling temperature of the solvent mixture. Alternatively, the reduction can be carried out by selective reduction of the nitro group in a manner known to the person skilled in the art, for example by hydrogen transfer reaction in the presence of a metal catalyst, for example palladium or preferably Raney nickel, in a suitable solvent, preferably a lower alcohol, using, for example ammonium formiate or preferably hydrazine hydrate as hydrogen donor.

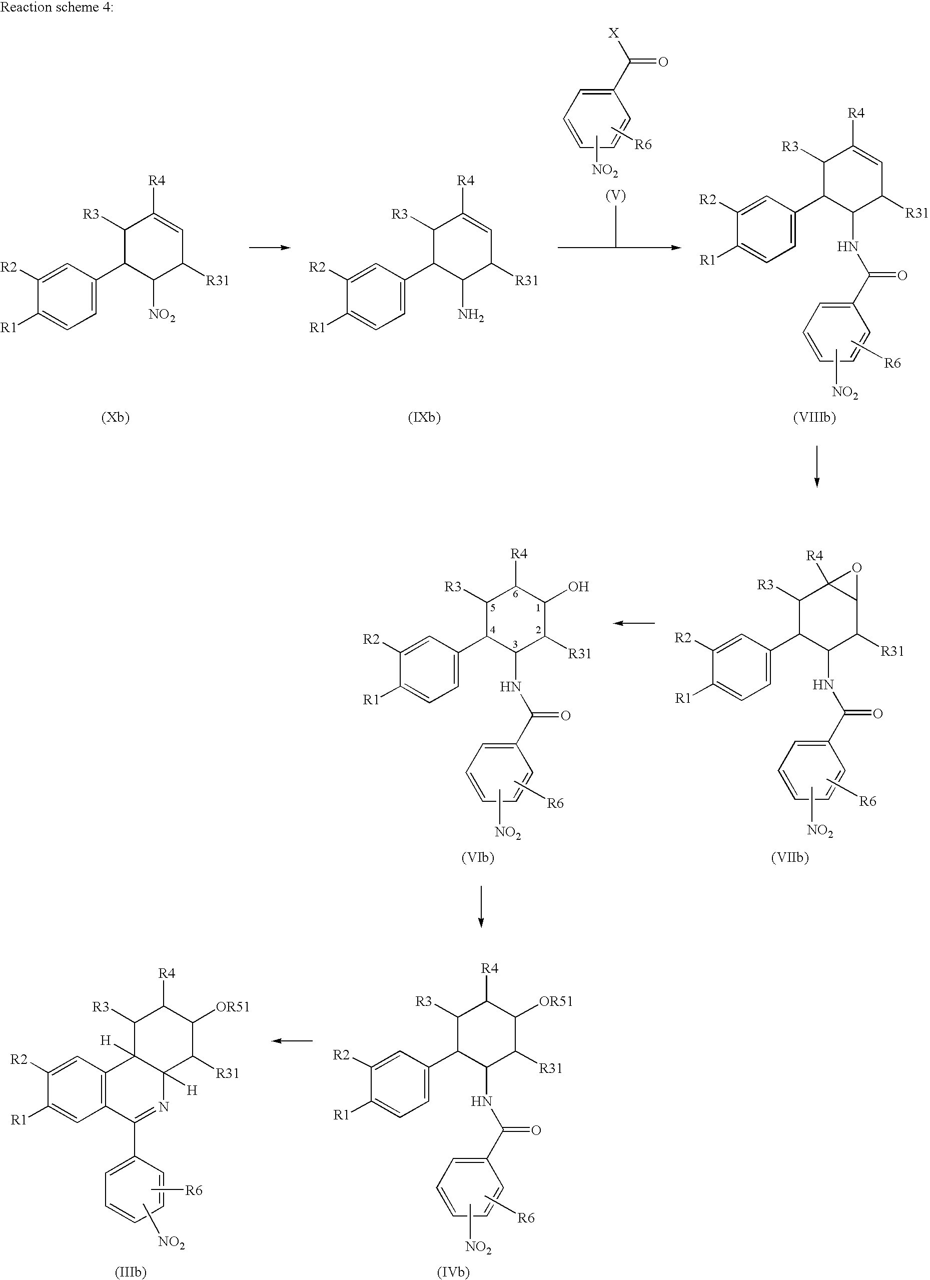

Compounds of the formula IXb obtained can be reacted, for example, as described by way of example in the following examples with compounds of the formula V, in which R6 has the meanings given above and X represents a suitable leaving group, preferably a chlorine atom, to give corresponding compounds of the formula VIIIb.

Alternatively, compounds of the formula VIIIb, in which R1, R2, R3, R31, R4 and R6 have the meanings given above in embodiment b, can also be prepared, for example, from corresponding compounds of the formula IXb and corresponding compounds of the formula V, in which X is hydroxyl, by reaction with amide bond linking reagents known to the person skilled in the art. Exemplary amide bond linking reagents known to the person skilled in the art which may be mentioned are, for example, the carbodiimides (e.g. dicyclohexylcarbodiimide or, preferably, 1-ethyl-3-(3-dimethylaminopropyl)carbodiimide hydrochloride), azodicarboxylic acid derivatives (e.g. diethyl azodicarboxylate), uronium salts [e.g. O-(benzotriazol-1-yl)-N,N,N′,N′-tetramethyluronium tetrafluoroborate or O-(benzotriazol-1yl)-N,N,N′,N′-tetramthyl-uronium-hexafluorophosphate] and N,N′-carbonyldiimidazole. In the scope of this invention preferred amide bond linking reagents are uronium salts and, particularly, carbodiimides, preferably, 1-ethyl-3-(3-dimethylaminopropyl)carbodiimide hydrochloride.

In the next step compounds of the formula VIIIb are converted into corresponding compounds of the formula VIIb by epoxidation reaction, which can be carried out as described in the following examples or in a manner known to one of ordinary skill in the art employing, for example, suitable epoxidation methods or suitable epoxidation reagents such as, for example, peracids (e.g. m-chloroperbenzoic acid) or organic or inorganic peroxides (e. g. dimethyldioxirane, hydrogene peroxide or persulfates).

Compounds of the formula VIIb obtained can be reduced by art-known methods to corresponding compounds of the formula VIb. More specifically, said reduction reaction can be performed employing, for example, as described by way of example in the following examples sodium borohydride as reductant. Alternatively, said reduction reaction can be also carried out using, for example, lithium aluminium hydride or a reductive mixture comprising noble metals, such as platinium dioxide or palladium, and a suitable hydrogen donor. With the aid of each of those said reduction methods, compounds of the formula Vb can be converted largely regio- and diastereoselectively into compounds of the formula IVb, wherein the hydroxyl radical in position 1 and the amido radical in position 3 are located at the same side of the plane defined by the cyclohexane ring.

It is moreover known to one of ordinary skill of the art, that the absolute configuration of a chiral carbon atom, preferably, to which a hydroxyl group and a hydrogen atom are bonded, can be inverted. Thus the configuration of the carbon atom in position 1 of compounds of the formula VIb can be optionally inverted. Said inversion of configuration of position 1 of compounds of the formula VIb can be achieved in a manner familiar to the person skilled in the art, for example by derivatization of position 1 with a suitable leaving group and subsequent replacement of said leaving group by a suitable nucleophile in a nucleophilic substitution reaction according to SN2 mechanism. Alternatively, said inversion of configuration of position 1 of compounds of the formula VIb can be also obtained, for example, as described by way of example in the following examples according to subsequently specified two step procedure shown in reaction scheme 5 below. In more detail, in the first step of said procedure shown in reaction scheme 5, exemplary compounds of the formula VIb*, in which R1, R2 and R6 have the meanings indicated above in embodiment b, and R3, R31 and R4 are hydrogen and position 1 has the R configuration, are converted by oxidation reaction into corresponding compounds of the formula XIb. Said oxidation is likewise carried out under conditions customary per se using, for example, chloranil, atmospheric oxygen, manganese dioxide or, preferably, chromium oxides as an oxidant. Then in the second step, compounds of the formula XIb obtained are converted by art-known reduction reaction of the keto group, preferably with metal hydride compounds or, more specifically, metal borohydrides, such as, for example, sodium borohydride, into corresponding compounds of formula VIb**, in which position 1 has now S configuration and thus the configuration of the carbon atom in position 1 is now inverted regarding to said compounds of the formula VIb*.

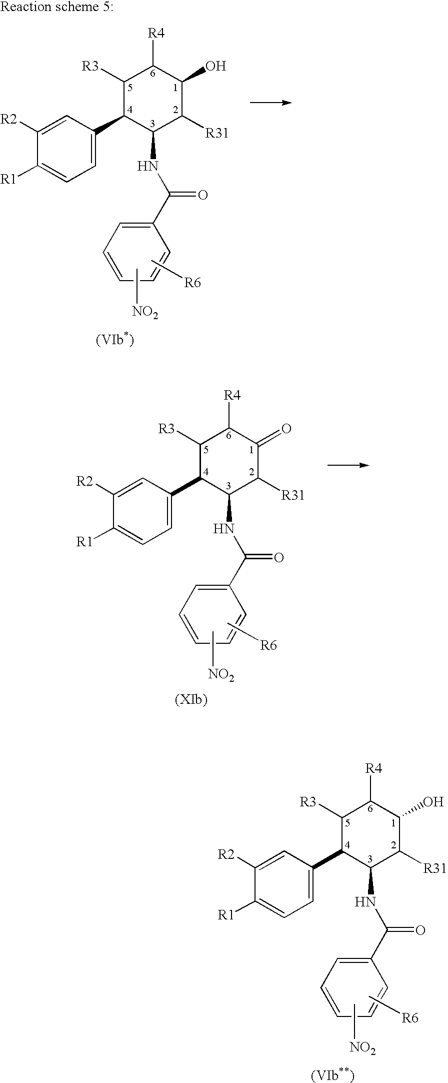

In the next reaction step of the synthesis route shown in reaction scheme 4 shown above, compounds of the formula VIb are converted into corresponding compounds of the formula IVb by introduction of the group R51 whereby R51 is other than hydrogen. The introduction reaction is carried out in a manner habitual per se (e.g. via alkylation or acylation reaction) or as described by way of example in the following examples.

The cyclization reaction leading to compounds of the formula IIlb, in which R1, R2, R3, R31, R4, R51 and R6 have the meanings given above in embodiment b whereby R51 is other than hydrogen, can be carried out, for example, as described by way of example in the following examples or analogously or similarly thereto, or as mentioned above for compounds according to embodiment a.

Compounds of the formula Xb, in which R1, R2, R3, R31 and R4 have the meanings mentioned above in embodiment b, are either known or can be obtained, for example as shown in reaction scheme 6, by the reaction of compounds of the formula IXa, in which R1 and R2 have the abovementioned meanings, with compounds of the formula XIIb, in which R3, R31 and R4 have the meanings indicated above in embodiment b.

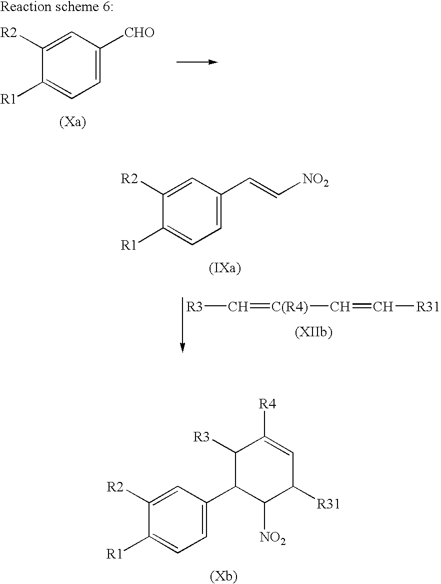

The cycloaddition is in this case carried out in a manner known to the person skilled in the art according to Diels-Alder, e.g. as described in J. Amer. Chem. Soc. 1957, 79, 6559 or in J. Org. Chem. 1952, 17, 581 or as described in the following examples.

Compounds of the formula Xb, in which the phenyl ring and the nitro group are trans to one another, can be converted such as known to the person skilled in the art into the corresponding cis compounds, e.g. as described in J. Amer. Chem. Soc. 1957, 79, 6559 or as described in the following examples.

The compounds of the formula XIIb are either known or can be prepared in a known manner.

In an alternative, compounds of the formula IVb, in which R1, R2, R3, R31, R4, R51 and R6 have the meanings given above in embodiment b whereby R51 is other than hydrogen (particularly compounds of formula IVb, in which R1, R2, R51 and R6 have the meanings given above in embodiment b whereby R51 is other than hydrogen, and R3, R31 and R4 are all hydrogen) can also be obtained as shown in reaction scheme 7 and as described by way of example in the following examples.

In the first reaction step of the route outlined in reaction scheme 7, the amino group of compounds of the formula IXB is protected with an art-known protective group PG1, such as e.g. the tert-butoxycarbonyl group. The proteced compounds are subjected to hydroboration reaction to obtain over two steps compounds of formula XIIIb. Said hydroboration reaction is carried out as described in the following examples using an appropriate (hydro)borating agent, such as e.g. 9-BBN, isopinocampheylborane or the like, or, particularly, borane-tetrahydrofuran (H₃B-THF), advantageously at ambient temperature. The compounds obtained are then converted into compounds of the formula XIIIb by introduction of the group R51 whereby R51 is other than hydrogen in a manner analogously as described above.

In the next reaction step of the synthesis route shown in reaction scheme 7, compounds of formula XIIIb are converted into corresponding compounds of the formula IVb by deprotection of the protective group PG1 and amidification with compounds of the formula V. Said reactions are carried out in a manner habitual per se or as described in the specification of this invention or in the following examples.

If necessary, the product obtained via said hydroboration reaction or, suitably, the R51-substituted derivative thereof is purified from resulting stereo- and/or regioisomeric side products by methods known to the person skilled in the art, such as e.g. by chromatographic separation techniques.

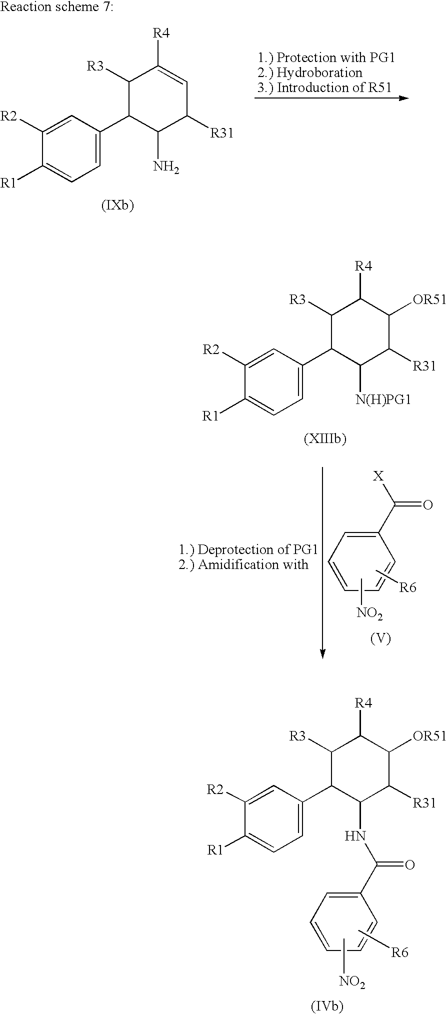

It is known to the person skilled in the art that if there are a number of reactive centers on a starting or intermediate compound it may be necessary to block one or more reactive centers temporarily by protective groups in order to allow a reaction to proceed specifically at the desired reaction center. A detailed description for the use of a large number of proven protective groups is found, for example, in T. Greene and P. Wuts, “Protective Groups in Organic Synthesis” (John Wiley & Sons, Inc. 1999, 3^{rd} Ed.) or in P. Kocienski, “Protecting Groups (Thieme Foundations Organic Chemistry Series N Group” (Thieme Medical Publishers, 2000).

The substances according to the invention are isolated and purified in a manner known per se, for example by distilling off the solvent under reduced pressure and recrystallizing the residue obtained from a suitable solvent or subjecting it to one of the customary purification methods, such as, for example, column chromatography on a suitable support material.

Salts are obtained by dissolving the free compound in a suitable solvent (e.g. a ketone, such as acetone, methyl ethyl ketone or methyl isobutyl ketone, an ether, such as diethyl ether, tetrahydrofuran or dioxane, a chlorinated hydrocarbon, such as methylene chloride or chloroform, or a low-molecular-weight aliphatic alcohol, such as ethanol or isopropanol) which contains the desired acid or base, or to which the desired acid or base is then added. The salts are obtained by filtering, reprecipitating, precipitating with a nonsolvent for the addition salt or by evaporating the solvent. Salts obtained can be converted into the free compounds, which can in turn be converted into salts, by alkalization or by acidification. In this manner, pharmacologically unacceptable salts can be converted into pharmacologically acceptable salts.

Suitably, the conversions mentioned in this invention can be carried out analogously or similarly to methods which are familiar per se to the person skilled in the art.

The person skilled in the art knows on the basis of his/her knowledge and on the basis of those synthesis routes, which are shown and described within the description of this invention, how to find other possible synthesis routes for compounds of the formula 1. All these other possible synthesis routes are also part of this invention.

Having described the invention in detail, the scope of the present invention is not limited only to those described characteristics or embodiments. As will be apparent to persons skilled in the art, modifications, analogies, variations, derivations, homologisations and adaptations to the described invention can be made on the base of art-known knowledge and/or, particularly, on the base of the disclosure (e.g. the explicite, implicite or inherent disclosure) of the present invention without departing from the spirit and scope of this invention as defined by the scope of the appended claims.

The following examples serve to illustrate the invention further without restricting it. Likewise, further compounds of the formula I, whose preparation is not explicitly described, can be prepared in an analogous or similar manner or in a manner familiar per se to the person skilled in the art using customary process techniques.

Any or all of the compounds which are mentioned in the following examples as final compounds as well as their salts, N-oxides and salts of the N-oxides are a preferred subject of the present invention.

In the examples, m.p. stands for melting point, h for hour(s), min for minutes, R_{f} for rentention factor in thin layer chromatography, s.p. for sintering point, EF for empirical formula, MW for molecular weight, MS for mass spectrum, M for molecular ion, fnd. for found, calc. for calculated, other abbreviations have their meanings customary per se to the skilled person.

According to common practice in stereochemistry, the symbols RS and SR are used to denote the specific configuration of each of the chiral centers of a racemate. In more detail, for example, the term “(2RS,4aRS,10bRS)” stands for a racemate (racemic mixture) comprising the one enantiomer having the configuration (2R,4aR,10bR) and the other enantiomer having the configuration (2S,4aS,10bS).

### EXAMPLES

### Final Compounds

### 1. N-[3-((2RS,4aRS,10bRS)9-Ethoxy-2-hydroxy-8-methoxy-1,2,3,4,4a,10b-hexahydro-phenanthridin-6-yl)-phenyl]-2-methoxy-acetamide

335 mg of acetic acid (2RS,4aRS,10bRS)-9-ethoxy-8-methoxy-6-[3-(2-methoxy-ethanoylamino)-phenyl]-1,2,3,4,4a,10b-hexahydro-phenanthridin-2-yl ester (Example 20) are dissolved in 1 ml of dichloromethane and 5 ml of methanol. 114 mg of cesium carbonate are added and the solution stirred for 19 h. The reaction mixture is adsorbed to silica gel and purified by flash chromatography to give 278 mg of the title compound.

EF: C₂₅H₃₀N₂O₅; MW: calc.: 438.53 MS: fnd.: 439.3 (MH⁺)

Starting from the appropriate ester compounds, which are mentioned or described explicitly below (compounds 20 to 38), or which can be prepared in a manner known to the person skilled in the art or analogously or similarly to the examples described herein, the following and also further relevant, non-explicitly described similar compounds are obtained according to the procedure as in Example 1.

### 2. N-[4-((2RS,4aRS,10bRS)-9-Ethoxy-2-hydroxy-8-methoxy-1,2,3,4,4a,10b-hexahydro-phenanthridin-6-yl)-phenyl]-3-methoxy-propionamide

EF: C₂₆H₃₂N₂O₅; MW: calc.: 452.56 MS: fnd.: 453.3 (MH⁺)

### 3. Cyclopropanecarboxylic acid [3-((2RS,4aRS,10bRS)-9-ethoxy-2-hydroxy-8-methoxy-1,2,3,4,4a,10b-hexahydro-phenanthridin-6-yl)-phenyl]-amide

EF: C₂₆H₃₀N₂O₄; MW: calc.: 434.54 MS: fnd.: 435.3 (MH⁺)

### 4. N-[4-((2RS,4aRS,10bRS)-9-Ethoxy-2-hydroxy-8-methoxy-1,2,3,4,4a,10b-hexahydro-phenanthridin-6-yl)-phenyl]-3-piperidin-1-yl-propionamide

EF: C₃₀H₃₉N₃O₄; MW: calc.: 505.66 MS: fnd.: 506.4 (MH⁺)

### 5. N-[4-((2RS,4aRS,10bRS)-9-Ethoxy-2-hydroxy-8-methoxy-1,2,3,4,4a,10b-hexahydro-phenanthridin-6-yl)-phenyl]-N′,N′-dimethyl-succinamide

EF: C₂₈H₃₅N₃O₅; MW: calc.: 493.61 MS: fnd.: 494.3 (MH⁺)

### 6. 1-Methyl-piperidine-4-carboxylic acid [4-((2RS,4aRS,10bRS)-9-ethoxy-2-hydroxy-8-methoxy-1,2,3,4,4a,10b-hexahydro-phenanthridin-6-yl)-phenyl]-amide

EF: C₂₉H₃₇N₃O₄; MW: calc.: 491.64 MS: fnd.: 492.4 (MH⁺)

### 7. Cyclopropanecarboxylic acid [4-((2RS,4aRS,10bRS)-9-ethoxy-2-hydroxy-8-methoxy-1,2,3,4,4a,10b-hexahydro-phenanthridin-6-yl)-phenyl]-amide

EF: C₂₆H₃₀N₂O₄; MW: calc.: 434.54 MS: fnd.: 435.3 (MH⁺)

### 8. N-[3-((2RS,4aRS,10bRS)-9-Ethoxy-2-hydroxy-8-methoxy-1,2,3,4,4a,10b-hexahydro-phenanthridin-6-yl)-phenyl]-3-methoxy-propionamide

EF: C₂₆H₃₂N₂O₅; MW: calc.: 452.56 MS: fnd.: 453.3 (MH⁺)

### 9. N-[4-((2RS,4aRS,10bRS)-9-Ethoxy-2-hydroxy-8-methoxy-1,2,3,4,4a,10b-hexahydro-phenanthridin-6-yl)-phenyl]-2-methoxy-acetamide

EF: C₂₅H₃₀N₂O₅; MW: calc.: 438.53 MS: fnd.: 439.2 (MH⁺)

### 10. N-[3-((2RS,4aRS,10bRS)-9-Ethoxy-2-hydroxy-8-methoxy-1,2,3,4,4a,10b-hexahydro-phenanthridin-6-yl)-phenyl]-3-piperidin-1-yl-propionamide

EF: C₃₀H₃₉N₃O₄; MW: calc.: 505.66 MS: fnd.: 506.3 (MH⁺)

### 11. 1-Methyl-piperidine-4-carboxylic acid [3-((2RS,4aRS,10bRS)-9-ethoxy-2-hydroxy-8-methoxy-1,2,3,4,4a,10b-hexahydro-phenanthridin-6-yl)-phenyl]-amide

EF: C₂₉H₃₇N₃O₄; MW: calc.: 491.64 MS: fnd.: 492.4 (MH⁺)

### 12. N-[3-((2RS,4aRS,10bRS)-9-Ethoxy-2-hydroxy-8-methoxy-1,2,3,4,4a,10b-hexahydro-phenanthridin-6-yl)-phenyl]-N′,N′-dimethyl-succinamide

EF: C₂₈H₃₅N₃O₅; MW: calc.: 493.61 MS: fnd.: 494.3 (MH⁺)

### 13. Dimethylamino-N-[4-((2RS,4aRS,10bRS)-9-ethoxy-2-hydroxy-8-methoxy-1,2,3,4,4a, 10b-hexahydro-phenanthridin-6-yl)-phenyl]-butyramide

### 14. Dimethylamino-N-[3-((2RS,4aRS,10bRS)-9-ethoxy-2-hydroxy-8-methoxy-1,2,3,4,4a, 10b-hexahydro-phenanthridin-6-yl)-phenyl]-butyramide

EF: C₂₈H₃₇N₃O₄; MW: calc.: 459.55 MS: fnd.: 460.2 (MH⁺)

### 15. 2-Acetylamino-N-[3-((2RS,4aRS,10bRS)-9-ethoxy-2-hydroxy-8-methoxy-1,2,3,4,4a, 10b-hexahydro-phenanthridin-6-yl)-phenyl]-acetamide

EF: C₂₅H₃₁N₃O₅; MW: calc.: 479.62 MS: fnd.: 480.3 (MH⁺)

### 16. 5-Oxo-pyrrolidine-2-carboxylic acid [3-((2RS,4aRS,10bRS)-9-ethoxy-2-hydroxy-8-methoxy-1,2,3,4,4a,10b-hexahydro-phenanthridin-6-yl)-phenyl]-amide

EF: C₂₇H₃₁N₃O₅; MW: calc.: 477.57 MS: fnd.: 478.3 (MH⁺)

### 17. N-[3-((2RS,4aRS,10bRS)-9-Ethoxy-2-hydroxy-8-methoxy-1,2,3,4,4a,10b-hexahydro-phenanthridin-6-yl)-phenyl]-2,6-dimethoxy-nicotinamide

EF: C₃₀H₃₃N₃O₆; MW: calc.: 531.61 MS: fnd.: 532.3 (MH⁺)

### 18. (2RS,4aRS,10bRS)-6-[3-(3-carbamoyl-propanoylamino)-phenyl]-9-ethoxy-8-methoxy 1,2,3,4,4a,10b-hexahydro-phenanthridin-2-ol

### 19. N-[3-((2RS,4aRS,10bRS)-9-Ethoxy-2-hydroxy-8-methoxy-1,2,3,4,4a,10b-hexahydro-phenanthridin-6-yl)-phenyl]-3-ureido-propionamide

EF: C₂₆H₃₂N₄O₅; MW: calc.: 480.57 MS: fnd.: 481.3 (MH⁺)

### 20. Acetic acid (2RS,4aRS,10bRS)-9-ethoxy-8-methoxy-6-[3-(2-methoxy-ethanoylamino)-phenyl]-1,2,3,4,4a,10b-hexahydro-phenanthridin-2-yl ester

216 mg of methoxyacetic acid and 460 mg of N-ethyl-N′-(3-dimethylaminopropyl)carbodiimide hydrochloride (EDCl) are placed in a flask. 2 mg of 4-dimethylaminopyridine and 1.2 mmol of acetic acid (2RS,4aRS,10bRS)-9-ethoxy-8-methoxy-6-(3-amino-phenyl)-1,2,3,4,4a,10b-hexahydro-phenanthridin-2-yl ester (compound A1) (both as solution in acetonitril) are added and the reaction is stirred at 70° C. for 16 h. The solvent is removed, the residue redissolved in 5 ml of dichloromethane and extracted with 5 ml of water. After reextraction of the water layer with dichloromethane the combined dichloromethane-layers are extracted with saturated sodium hydrogencarbonate solution. The organic layer is dried over sodium sulfate and the solvent removed. After purification by flash chromatography 417 mg of the title compound are obtained as a yellowish foam.

EF: C₂₇H₃₂N₂O₆; MW: calc.: 480.57 MS: fnd.: 481.3 (MH⁺)

Starting from the appropriate carboxylic acids, which are known or accessible via known procedures, and the appropriate amino starting compounds, which are mentioned or described explicitly below, or which can be prepared in a manner known to the person skilled in the art or analogously or similarly to the examples described herein, the following and also further relevant, non-explicitly described similar compounds are obtained according to the procedure as in Example 20.

### 21. Acetic acid (2RS,4aRS,10bRS)-9-ethoxy-8-methoxy-6-[4(3-methoxy-propanoylamino)-phenyl]-1,2,3,4,4a,10b-hexahydro-phenanthridin-2-yl ester

EF: C₂₈H₃₄N₂O₆; MW: calc.: 494.59 MS: fnd.: 495.3 (MH⁺)

### 22. Acetic acid (2RS,4aRS,10bRS)-6-{3-[(1-cyclopropyl-methanoyl)amino]-phenyl}-9-ethoxy-8-methoxy-1,2,3,4,4a,10b-hexahydro-phenanthridin-2-yl ester

EF: C₂₈H₃₂N₂O₅; MW: calc.: 476.58 MS: fnd.: 477.3 (MH⁺)

### 23. Acetic acid (2RS,4aRS,10bRS)-9-ethoxy-8-methoxy-6-[4-(3-piperidin-1-yl-propanoylamino)-phenyl]-1,2,3,4,4a,10b-hexahydro-phenanthridin-2-yl ester

EF: C₃₂H₄₁N₃O₅; MW: calc.: 547.7 MS: fnd.: 548.3 (MH⁺)

### 24. Acetic acid (2RS,4aRS,10bRS)-6-[4-(3-dimethylcarbamoyl-propanoylamino)-phenyl]-9-ethoxy-8-methoxy-1,2,3,4,4a,10b-hexahydro-phenanthridin-2-yl ester

EF: C₃₀H₃₇N₃O₆; MW: calc.: 535.65 MS: fnd.: 536.2 (MH⁺)

### 25. Acetic acid (2RS,4aRS,10bRS)-9-ethoxy-8-methoxy-6-(4-{[1-(1-methyl-piperidin-4-yl)-methanoyl]-amino}-phenyl)-1,2,3,4,4a,10b-hexahydro-phenanthridin-2-yl ester

EF: C₃₁H₃₉N₃O₅; MW: calc.: 533.67 MS: fnd.: 534.4 (MH⁺)

### 26. Acetic acid (2RS,4aRS,10bRS)-6-{4-[(1-cyclopropyl-methanoyl)-amino]-phenyl }-9-ethoxy-8-methoxy-1,2,3,4,4a,10b-hexahydro-phenanthridin-2-yl ester

EF: C₂₈H₃₂N₂O₅; MW: calc.: 476.58 MS: fnd.: 477.4 (MH⁺)

### 27. Acetic acid (2RS,4aRS,10bRS)-9-ethoxy-8-methoxy-6-[3-(3-methoxy-propanoylamino)-phenyl]-1,2,3,4,4a,10b-hexahydro-phenanthridin-2-yl ester

EF: C₂₈H₃₄N₂O₆; MW: calc.: 494.59 MS: fnd.: 495.3 (MH⁺)

### 28. Acetic acid (2RS,4aRS,10bRS)-9-ethoxy-8-methoxy-6-[4-(2-methoxy-ethanoylamino)-phenyl]-1,2,3,4,4a,10b-hexahydro-phenanthridin-2-yl ester

EF: C₂₇H₃₂N₂O₆; MW: calc.: 480.57 MS: fnd.: 481.3 (MH⁺)

### 29. Acetic acid (2RS,4aRS,10bRS)-9-ethoxy-8-methoxy-6-[3-(3-piperidin-1-yl-propanoylamino)-phenyl]-1,2,3,4,4a,10b-hexahydro-phenanthridin-2-yl ester

EF: C₃₂H₄₁N₃O₅; MW: calc.: 547.7 MS: fnd.: 548.3 (MH⁺)

### 30. Acetic acid (2RS,4aRS,10bRS)-9-ethoxy-8-methoxy-6-(3-{[1-(1-methyl-piperidin-4-yl)-methanoyl]-amino}-phenyl)-1,2,3,4,4a,10b-hexahydro-phenanthridin-2-yl ester

EF: C₃₁H₃₉N₃O₅; MW: calc.: 533.67 MS: fnd.: 534.4 (MH⁺)

### 31. Acetic acid (2RS,4aRS,10bRS)-6-[3-(3-dimethylcarbamoyl-propanoylamino)-phenyl]-9-ethoxy-8-methoxy-1,2,3,4,4a,10b-hexahydro-phenanthridin-2-yl ester

EF: C₃₀H₃₇N₃O₆; MW: calc.: 535.65 MS: fnd.: 534.3 (MH⁺)

### 32. Acetic acid (2RS,4aRS,10bRS)-6-[4(4-dimethylamino-butanoylamino)-phenyl]-9-ethoxy-8-methoxy-1,2,3,4,4a,10b-hexahydro-phenanthridin-2-yl ester

EF: H₃₀H₃₉N₃O₅; MW: calc.: 521.66 MS: fnd.: 522.3 (MH⁺)

### 33. Acetic acid (2RS,4aRS,10bRS)-6-[3-(4-dimethylamino-butanoylamino)-phenyl]-9-ethoxy-8-methoxy-1,2,3,4,4a,10b-hexahydro-phenanthridin-2-yl ester

EF: C₃₀H₃₉N₃O₅; MW: calc.: 521.66 MS: fnd.: 522.4 (MH⁺)

### 34. Acetic acid (2RS,4aRS,10bRS)-6-[3-(2-acetylamino-ethanoylamino)-phenyl]-9-ethoxy-8-methoxy-1,2,3,4,4a,10b-hexahydro-phenanthridin-2-yl ester

EF: C₂₈H₃₃N₃O₆; MW: calc.: 507.59 MS: fnd.: 508.3 (MH⁺)

### 35. Acetic acid (2RS,4aRS,10bRS)-9-ethoxy-8-methoxy-6-(3-{[1-(5-oxo-pyrrolidin-2-yl)-methanoyl]-amino}-phenyl)-1,2,3,4,4a,10b-hexahydro-phenanthridin-2-yl ester

EF: C₂₉H₃₃N_{3 O}₆; MW: calc.: 519.6 MS: fnd.: 520.3 (MH⁺)

### 36. Acetic acid (2RS,4aRS,10bRS)-6-(3-{[1-(2,6dimethoxy-pyridin-3-yl)-methanoyl]-amino}-phenyl)-9-ethoxy-8-methoxy-1,2,3,4,4a,10b-hexahydro-phenanthridin-2-yl ester

EF: C₃₂H₃₅N₃O₇; MW: calc.: 573.65 MS: fnd.: 574.3 (MH⁺)

### 37. Acetic acid (2RS,4aRS,10bRS)-6-[3-(3-carbamoyl-propanoylamino)-phenyl]-9-ethoxy-8-methoxy-1,2,3,4,4a,10b-hexahydro-phenanthridin-2-yl ester

EF: C₂₈H33N₃O₆; MW: calc.: 507.59 MS: fnd.: 508.3 (MH⁺)

### 38. Acetic acid (2RS,4aRS,10bRS)-9-ethoxy-8-methoxy-6-[3-(3-ureido-propanoylamino)-phenyl]-1,2,3,4,4a,10b-hexahydro-phenanthridin-2-yl ester

EF: C₂₈H₃₄N₄O₆; MW: calc.: 522.61 MS: fnd.: 523.3 (MH⁺) 39. Acetic acid (3SR,4aRS,10bRS)-6-{3-[(1-cyclopropyl-methanoyl)-amino]-phenyl}-9-ethoxy-8-methoxy-1,2,3,4,4a,10b-hexahydro-phenanthridin-3-yl ester

EF: C₂₈H₃₂N₂O₅; MW: calc.: 476.58 MS: fnd.: 477.3 (MH⁺)

### 40. Cyclopropanecarboxylic acid [3-((3SR,4aRS,10bRS)-9-ethoxy-3-hydroxy-8-methoxy-1,2,3,4,4a,10b-hexahydro-phenanthridin-6-yl)-phenyl]-amide

Starting from acetic acid (3SR,4aRS,10bRS)-6-{3-[(1-cyclopropyl-methanoyl)-amino]-phenyl}-9-ethoxy-8-methoxy-1,2,3,4,4a,10b-hexahydro-phenanthridin-3-yl ester (compound 39) the title compound is obtained according to the procedure as in Example 1.

EF: C26H30N2O4; MW: calc.: 434.54 MS: fnd.: 435.3 (MH⁺)

Starting Compounds

### A1. (2RS,4aRS,10bRS)-9-Ethoxy-8-methoxy-6-(3-amino-phenyl-1,2,3,4,4a,10b-hexahydro-phenanthridin-2-yl ester

219 mg of (2RS,4aRS,10bRS)-9-ethoxy-8-methoxy-6-(3-nitro-phenyl)-1,2,3,4,4a,10b-hexahydro-phenanthridin-2-yl ester (compound B1) and 564 mg tin dichloride dihydrate (SnCl₂.2H₂O) are dissolved in 3 ml of ethanol and heated for 45 min. under reflux. The solvent is removed and the residue is dissolved in ethyl acetate and saturated aqueous sodium hydrogencarbonate solution. After reextraction with ethylacetate the organic layer is dried over sodium sulfate. The solvent is removed to give 198 mg of the title compound.

Starting from the appropriate starting compounds, which are mentioned or described explicitly below, or which can be prepared in a manner known to the person skilled in the art or analogously or similarly to the examples described herein, further relevant, non-explicitly described compounds, which are analogues to compound A1, are obtained according to the procedure as in Example A1 or analogously or similarly thereto under conditions appropriate desired reactions to occur.

### B1. (2RS,4aRS,10bRS)-9-Ethoxy-8-methoxy-6-(3-nitro-phenyl)-1,2,3,4,4a,10b-hexahydro-phenanthridin-2-yl ester

744 mg of acetic acid (1RS,3RS,4RS)-4-amino-3-(3-ethoxy-4-methoxy-phenyl)-cyclohexyl ester (compound C1), 485 mg of 3-nitrobenzoic acid and 557 mg of N-ethyl-N′-(3-dimethylaminopropyl)carbodiimide hydrochloride (EDCl) are dissolved in 5ml of dichloromethane, 2 mg of 4-dimethylaminopyridine are added and stirred for 16 h. 5 ml of water are added and the organic layer extracted with saturated aqueous sodium hydrogencarbonate solution. After drying the organic layer with sodium sulfate and removing the solvent the crude product is purified by flash chromatography and directly used for the following cyclisation step:

The product from above is dissolved in 4 ml of phosphorus oxychloride and heated at 100° C. for 6 h. After cooling to room temperature 5ml of dichloromethane are added and the mixture poured into 3 M aqueous sodium hydroxide solution. Water is added to dissolve the precipitating solid and the pH is adjusted to pH>10. The organic layer is dried over sodium sulfate. The crude product is purified by flash chromatography on silica to yield 813 mg of the title compound.

Starting from the appropriate starting compounds, which are mentioned or described explicitly below, or which can be prepared in a manner known to the person skilled in the art or analogously or similarly to the examples described herein, further relevant, non-explicitly described compounds, which are analogues to compound B1, are obtained according to the procedure as in Example B1 or analogously or similarly thereto under conditions appropriate desired reactions to occur. If necessary, the abovementioned cyclization is carried out in the presence of a suitable Lewis Acid, such as e.g. tin tetrachloride.

### C1. Acetic acid (1RS,3RS,4RS)-4amino-3-(3-ethoxy-4-methoxy-phenyl)-cyclohexyl ester

Starting from compound D1 mentioned below, the title compound is obtained analogously to the procedure as in Example C2.

EF: C₁₇H₂₅NO₄; MW: 307.39 MS: 308.0 (MH⁺)

### C1a. Acetic acid (1R,3R,4R)-4-amino-3-(3-ethoxy-4-methoxy-phenyl)-cyclohexyl ester

24.0 g (55.0 mmol) of the pyroglutamate of the title compound (compound B1b) are suspended in 150 ml of water, 100 ml of dichloromethane are added, then saturated KHCO₃-solution until the gas evolution ceased. After phase separation, reextraction of the water layer and drying the combined organic layers with sodium sulfate the solvent is removed to give 16.9 g of the salt-free title compound.

Analytical Column Chromatography (CHIRALPAK AD-H 250×4.6 mm 5μ No.ADH0CE-DB030, Eluent: n-Hexan/iPrOH=80/20 (v/v)+0.1% Diethylamine): Retention Time: 6.54 min

### C1b. Acetic acid (1R,3R,4R)-4-amino-3-(3-ethoxy-4-methoxy-phenyl)-cyclohexyl ester, salt with L-pyroglutamic acid

Solution A: 55.2 g (180 mmol) of racemic acetic acid (1RS,3RS,4RS)-4-amino-3-(3-ethoxy-4-methoxy-phenyl)-cyclohexyl ester (compound B1) are dissolved in 540 ml of isopropyl acetate.

Solution B: 18.6 g (144 mmol) of L-pyroglutamic acid are dissolved in 260 ml of isopropanol under heating, then 290 ml of isopropyl acetate is added carefully.

Solution B is added to solution A and left for 48 hours. The solid is filtered off and washed with a little isopropyl acetate to give after drying 32.48 g colorless crystals with a ratio of the enantiomers of 97:3 in favour of the title compound.

M.p.: 165-167° C.

### C2. Acetic acid (1RS,3RS,4RS)-4-amino-3-(3,4dimethoxyphenyl)cyclohexyl ester

A solution of 10.37 g of acetic acid (1RS,3RS,4RS)-3-(3,4-dimethoxyphenyl)-4-nitrocyclohexyl ester (compound D2) in 240 ml of ethanol is added to a zinc-copper couple, prepared from 16.8 g of zinc powder and 920 mg of copper (II) acetate monohydrate in acetic acid, the resulting suspension is refluxed and treated with 26 ml of acetic acid, 3.2 ml of water and 26 ml of ethanol. The resulting mixture is refluxed for further 15 min. The precipitate is filtered off with suction and the solvent is removed. Chromatographical purification on silica gel using a mixture of petroleum ether/ethyl acetate/triethylamine in the ratio 2/7/1 and concentration of the corresponding eluate fractions afford 5.13 g (55% of theory) of the title compound as a pale brown oil.

R_{f}=0.35 (petroleum ether/ethyl acetate/triethylamine=2/7/1)

### C3. Acetic acid (1RS,3RS,4RS)-4-amino-3-[4-(1,1-difluoro-methoxy)-3-methoxy-phenyl]-cyclohexyl ester

Starting from compound D3 mentioned below, the title compound is obtained according to the procedure as in Example C2.

EF: C₁₆H₂₁F₂NO₄; MW: 329.35 MS: 330.0 (MH⁺)

### C4. Acetic acid (1RS,3RS,4RS)-4-amino-3-[3-(1,1-difluoro-methoxy)-4-methoxy-phenyl]-cyclohexyl ester

Starting from compound D4 mentioned below, the title compound is obtained according to the procedure as in Example C2.

EF: C₁₆H₂₁F₂NO₄; MW: 329.35 MS: 330.0 (MH⁺)

### C5. Acetic acid (1RS,3RS,4RS)-4-amino-3-[3-(2,2-difluoro-ethoxy)-4-methoxy-phenyl]-cyclohexyl ester

Starting from compound D5 mentioned below, the title compound is obtained according to the procedure as in Example C2.

### C5a. Acetic acid (1R,3R,4R)-4-amino-3-[3-(2,2-difluoro-ethoxy)-4-methoxy-phenyl]-cyclohexyl ester

The title compound is obtained from its pyroglutamate salt (compound C5b) analogously as described for compound C1a using sodium hydrogencarbonate solution.

### C5b. Acetic acid (1R,3R,4R)-4-amino-3-[3-(2,2-difluoro-ethoxy)-4-methoxy-phenyl]-cyclohexyl ester, salt with L-pyroglutamic acid

343 mg (1.00 mmol) of acetic acid (1RS,3RS,4RS)-4-amino-3-[3-(2,2-difluoro-ethoxy)-4-methoxy-phenyl]-cyclohexyl ester (compound C5) are dissolved in 3 ml of isopropanol. A solution of 103 mg (0.80 mmol) of L-pyroglutamic acid in 2 ml of isopropanol is added. After filtering and drying 162 mg of the pyroglutamate are isolated with an enantiomeric ratio of 97:3 in favour of the title compound.

### C6. Acetic acid (1SR,3RS,4RS)3-amino-4-(3-ethoxy-4-methoxy-phenyl)-cyclohexyl ester

3.0 g (7.36 mmol) of acetic acid (1SR,3RS,4RS)-3-tert-butoxycarbonylamino-4-(3-ethoxy-4-methoxy-phenyl)-cyclohexyl ester (compound D6) are dissolved in 6 ml of 4 M HCl in dioxane and stirred for 30 min. After removal of the solvent the residue is dissolved in dichloromethane and 25 ml of sat. NaHCO₃ solution are added carefully. After phase separation, reextraction of the water layer and drying of the combined organic layers (Na₂SO₄) the solvent is removed to give 2.25 g of the title compound.

EF: C17 H25 N O4; MW: 307.39 MS: 308.1 (MH⁺)

### C7. Acetic acid (1SR,3RS,4RS)-3-amino-4-(3,4dimethoxy-phenyl)-cyclohexyl ester

The title compound can be obtained from compound D7 analogously as described for compound C6.

### D1. Acetic acid (1RS,3RS,4RS)-3-(3-ethoxy-4-methoxy-phenyl)-4-nitrocyclohexyl ester

Starting from compound E1mentioned below, the title compound is obtained according to the procedure as in Example D2.

### D2. Acetic acid (1RS,3RS,4RS)-3-(3,4dimethoxyphenyl)-4-nitrocyclohexyl ester

10.18 g of (1RS,3RS,4RS)-3-(3,4dimethoxyphenyl)-4-nitrocyclohexanol (compound E2) are dissolved in 100 ml of acetic anhydride and the solution is heated to 100° C. for 1-2 h. After removal of the solvent, the residue is chromatographed on silica gel using a mixture of petroleum ether/ethyl acetate in the ratio 2/1. Concentration of the corresponding eluate fractions furnish 10.37 g (89% of theory) of the title compound as an oil.

R_{f}=0.32 (petroleum ether/ethyl acetate=2/1)

Starting from the starting compounds which are mentioned below or which can be obtained analogously to the described examples, the following are obtained according to the procedure as in Example D2:

### D3. Acetic acid (1RS,3RS,4RS)-3-[4-(1,1-difluoro-methoxy)-3-methoxy-phenyl]-4-nitrocyclohexyl ester

### D4. Acetic acid (1RS,3RS,4RS)-3-[3-(1,1-difluoro-methoxy)-4-methoxy-phenyl]-4-nitrocyclohexyl ester

### D5. Acetic acid (1RS,3RS,4RS)-3-[3-(2,2-difluoro-ethoxy)-4-methoxy-phenyl]-4-nitrocyclohexyl ester

### D6. Acetic acid (1SR,3RS,4RS)-3-tert-butoxycarbonylamino-4-(3-ethoxy-4-methoxy-phenyl)-cyclohexyl ester

22.64 g (65 mmol) of [(1RS,6RS)-6-(3-ethoxy-4-methoxy-phenyl)-cyclohex-3-enyl]-carbamic acid tert-butyl ester (compound E6) are dissolved in 180 ml of THF and 50 ml of BH₃ (1 M solution in THF) are added dropwise (30 min). After stirring for 2 h the mixture is cooled using an ice bath and a mixture of 30 ml of H₂O₂ (30%) and 60 ml of aqueous NaOH (3 M) is added. The mixture is stirred for 30 min at room temperature. 400 ml of water and 200 ml of dichloromethane are added. After phase separation, reextraction of the water layer and drying of the combined organic layers (Na₂SO₄) the solvent is removed and the crude product (23.42 g, mixture of the two mentioned regioisomers ˜2:1 in favour of the title compound) is used directly without further purification.

The crude material from above then is dissolved in 50 ml of pyridine. 50 mg of 4-dimethylaminopyridine and 60 ml of acetic anhydride are added and the mixture stirred for 90 min at 100° C. The solvents and the acetic anhydride are removed (sat. NaHCO₃ solution). Purification by means of chromatography yields 9.4 g of the title compound as colorless foam.

EF: C22 H33 N 06; MW: 407.51 MS: 308.1 (MH⁺-Boc), 407.8 (MH⁺), 430.1 (MNa⁺)

### D7. Acetic acid (1SR,3RS,4RS)-3-tert-butoxycarbonylamino-4-(3,4-dimethoxy-phenyl)-cyclohexyl ester

The title compound can be obtained from compound E7 analogously as described for compound D6.

### E1. (1RS,3RS,4RS)-3-(3-Ethoxy-4-methoxy-phenyl)-4-nitrocyclohexanol

Starting from compound F1 mentioned below, the title compound is obtained according to the procedure as in Example E2.

### E2. (1RS,3RS,4RS)-3-(3,4-Dimethoxyphenyl)-4-nitrocyclohexanol

10 g of (1RS,3RS,4SR)-3-(3,4-dimethoxyphenyl)-4-nitrocyclohexanol (compound F2) are dissolved in 170 ml of absolute 1,2-dimethoxyethane. 14.3 ml of a 30% solution of sodium methanolate in methanol are added dropwise. After complete addition, stirring is continued for 10 min and a mixture consisting of 85% phosphoric acid and methanol is added to pH 1. By adding of saturated potassium hydrogencarbonate solution the resulting suspension is neutralized. The mixture is diluted with water and dichloromethane, the organic layer is separated and extracted with dichloromethane. The solvents are removed under reduced pressure to yield the title compound as a pale yellow oil, which crystallizes. The title compound is used without further purification in the next step.

R_{f}=0.29 (petroleum ether/ethyl acetate=1/1) M.p.: 126-127° C.

Starting from the appropriate starting compounds mentioned below, the following are obtained according to the procedure as in Example E2:

### E3. (1RS,3RS,4RS)-3-[4-(1,1-Difluoro-methoxy)-3-methoxy-phenyl]-4nitrocyclohexanol

### E4. (1RS,3RS,4RS)-3-[3-(1,1-Difluoro-methoxy)-4-methoxy-phenyl]-4-nitrocyclohexano

### E5. (1RS,3RS,4RS)-3-[3-(2,2-Difluoro-ethoxy)-4-methoxy-phenyl]-4-nitrocyclohexanol

### E6. [(1RS,6RS)-6-(3-Ethoxy-4-methoxy-phenyl)-cyclohex-3-enyl]-carbamic acid tert-butyl ester

Starting from (1RS,6RS)-6-(3-ethoxy-4-methoxy-phenyl)-cyclohex-3-enylamine (compound F6) the title compound is obtained analogously as described for compound E7.

EF: C20 H29 N O4; MW: 347.46, MS: 370.1 (MNa⁺)

### E7. [(1RS,6RS)-6-(3,4Dimethoxy-phenyl)-cyclohex-3-enyl]-carbamic acid tert-butyl ester

15.18 g (65.06 mmol) of (±)-cis-6-(3,4-dimethoxyphenyl)-cyclohex-3-enylamine (compound F7) and 14.21 g (65.11 mmol) of Boc₂O are stirred in dichloromethane for 2.5 h, then the solvent is removed and the residue crystallized from ethylacetate/n-heptane to give 19.1 g of the title compound.

EF: C19 H27 N O4; MW: 333.43, MS: 334.2 (MH⁺)

### F1. (1RS,3RS,4SR)-3-(3-Ethoxy-4-methoxy-phenyl)-4-nitrocyclohexanol

Starting from compound G1 mentioned below, the title compound is obtained according to the procedure as in Example F2.

### F2. (1RS,3RS,4SR)-3-(3,4-Dimethoxyphenyl)-4-nitrocyclohexanol

Under nitrogen atmosphere 16.76 g of (3RS,4SR)-3-(3,4-dimethoxyphenyl)-4-nitrocyclohexanone (compound G2) are dissolved in 300 ml of tetrahydrofurane, the solution is cooled to −78° C., and 75 ml of 1 M solution of potassium tri-sec-butylborohydride in tetrahydrofurane is added dropwise. After stirring for further 1 h, a mixture consisting of 30% hydrogeneperoxide solution and phosphate buffer solution is added. Stirring is continued for further 10 min, the reaction mixture is diluted with 400 ml of ethyl acetate and the aqueous layer is extracted with ethyl acetate, the combined organic phases are concentrated to give a foam, which is purified by chromatography on silica gel using a mixture of petroleum ether/ethyl acetate in the ratio 1/1 to furnish 10.18 g (60% of theory) of the title compound.

EF: C₁₄H₁₉NO₅; MW: 281.31 MS: 299.1 (MNH₄⁺) R_{f}=0.29 (petroleum ether/ethyl acetate=1/1) M.p.: 139-141° C.

Starting from the appropriate starting compounds mentioned below, the following are obtained according to the procedure as in Example F2:

### F3. (1RS,3RS,4SR)-3-[4-(1,1-Difluoro-methoxy)-3-methoxy-phenyl]-4-nitrocyclohexanol

### F4. (1RS,3RS,4SR)-3-[3-(1,1-Difluoro-methoxy)-4-methoxy-phenyl]-4-nitrocyclohexanol

### F5. (1RS,3RS,4SR)-3-[3-(2,2-Difluoro-ethoxy)-4-methoxy-phenyl]-4-nitrocyclohexanol

### F6. (1RS,6RS)-6-(3-Ethoxy-4-methoxy-phenyl)-cyclohex-3-enylamine

Starting from 2-ethoxy-1-methoxy-4-((1RS,6RS)-6-nitro-cyclohex-3-enyl)-benzene (compound G6) the title compound is obtained analogously as described for compound F7.

### F7. (±)-cis-6-(3,4-Dimethoxyphenyl)-cyclohex-3-enylamine

40 g of (±)-cis-1,2-dimethoxy-4-(2-nitrocyclohex-4-enyl)benzene (compound G7) are dissolved in 400 ml of ethanol and 40 g of zinc powder are added. After heating to boiling temperature, 65 ml of glacial acetic acid are added dropwise. Afterwards, the reaction mixture is filtrated and concentrated. The residue is redissolved in diluted hydrochloric acid and extracted with toluene. The aqueous layer is alkalized using 6 N solution of sodium hydroxide and extracted several times with toluene. The combined organic phases of the alkalic extraction are dried using sodium sulfate and concentrated. The residue is chromatographed on silica gel. 11.5 g of the title compound are obtained.

### G1. (3RS,4SR)-3-(3-Ethoxy-4-methoxy-phenyl)-4-nitrocyclohexanone

Starting from compound H1 mentioned below, the title compound is obtained according to the procedure as in Example G2.

### G2. (3RS,4SR)-3-(3,4-Dimethoxyphenyl)-4-nitrocyclohexanone

90.0 g of 3,4-dimethoxy-ωnitrostyrene (compound H2), 90 ml of 2-trimethylsilyloxy-1,3-butadiene and 180 ml of abs. toluene are put in an autoclave, where the mixture is stirred at 140° C. for 2 days and then cooled. After addition of 1000 ml of ethyl acetate, 300 ml of a 2 N solution of hydrochloric acid are dropped under stirring. The phases are separated and the aqueous layer is extracted three times with dichloromethane. The combined organic extracts are washed with saturated sodium hydrogencarbonate solution, dried over magnesium sulfate and the solvents are removed under reduced pressure to give 150 g of the crude title compound. Further purification is carried out by chromatography on silica gel using petroleum ether/ethyl acetate in the ratio 1/1 as eluent to give 81.5 g (67% of theory) of the pure title compound.

EF: C₁₄H₁₇NO₅; MW: 279.30 MS: 279 (M⁺), 297.1 (MNH₄⁺) R_{f}=0.47 (petroleum ether/ethyl acetate=1/1) M.p.: 147-148° C.

Starting from the appropriate starting compounds mentioned below, the following are obtained according to the procedure as in Example G2:

### G3. (3RS,4SR)-3-[4-(1,1-Difluoro-methoxy)-3-methoxy-phenyl]-4-nitrocyclohexanone

### G4. (3RS,4SR)-3-[3-(1,1-Difluoro-methoxy)-4-methoxy-phenyl]-4-nitrocyclohexanone

### G5. (3RS,4SR)-3-[3-(2,2-Difluoro-ethoxy)-4-methoxy-phenyl]-4-nitrocyclohexanone

### G6. 2-Ethoxy-1-methoxy-4-((1RS,6RS)-6-nitro-cyclohex-3-enyl)-benzene

Starting from 2-ethoxy-1-methoxy-4-((1RS,6SR)-6-nitro-cyclohex-3-enyl)-benzene (compound H6) the title compound is obtained analogously as described for compound G7.

### G7. (±)-cis-1,2-Dimethoxy-4-(2-nitrocyclohex-4-enyl)benzene

10.0 g of (±)-trans-1,2-dimethoxy-4-(2-nitrocyclohex-4-enyl)benzene (compound H7) and 20.0 g of potassium hydroxide are dissolved in 150 ml of ethanol and 35 ml of dimethylformamide. A solution of 17.5 ml of conc. sulfuric acid in 60 ml of ethanol is then added dropwise such that the internal temperature does not exceed 4° C. After stirring for 1 h, the mixture is added to 1 l of ice water, the precipitate is filtered off with suction, washed with water and dried, and the crude product is recrystallized in ethanol. 8.6 g of the title compound of m.p. 82.5-84° C. are obtained.

### H1. 3-Ethoxy-4-methoxy-ω-nitrostyrene

Starting from art-known starting compounds, the title compound is obtained according to the procedure as in Example H2:

### H2. 3,4-Dimethoxy-ω-nitrostyrene

207.0 g of 3,4-dimethoxybenzaldehyde, 100.0 g of ammonium acetate and 125 ml of nitromethane are heated to boiling for 3-4 h in 1.0 l of glacial acetic acid. After cooling in an ice bath, the precipitate is filtered off with suction, rinsed with glacial acetic acid and petroleum ether and dried. M.p.: 140-141° C. Yield: 179.0 g.

Starting from starting compounds, which are art-known or which can be obtained according to known procedures, such as e.g. as described in WO 95/01338 or analogously or similarly thereto, the following compounds are obtained according to the procedure as in Example H2:

### H3. 4-(1,1-Difluoro-methoxy)-3-methoxy-ω-nitrostyrene

### H4. 3-(1,1-Difluoro-methoxy)-4-methoxy-ω-nitrostyrene

### H5. 3-(2,2-Difluoro-ethoxy)-4-methoxy-ω-nitrostyrene

The title compound is obtained starting from 3-(2,2-difluoro-ethoxy)-4-methoxy-benzaldehyde (compound I1) according to the procedure as in Example H2.

M.p.: 164-165° C.

### H6. 2-Ethoxy-1-methoxy-4-((1RS,6SR)-6-nitro-cyclohex-3-enyl)-benzene

Starting from 3-ethoxy-4-methoxy-ω-nitrostyrene (compound H1) the title compound is obtained analogously as described for compound H7.

### H7. (±)-trans-1,2-Dimethoxy-4-(2-nitrocyclohex-4-enyl)benzene

50.0 g of 3,4-dimethoxy-ω-nitrostyrene (compound H2), and 1.0 g (9.1 mmol) of hydroquinone are suspended in 200 ml of abs. toluene and treated at −70° C. with 55.0 g (1.02 mol) of liquid 1,3-butadiene. The mixture is stirred at 160° C. for 6 days in an autoclave and then cooled. Some of the solvent is removed on a rotary evaporator, and the resulting precipitate is filtered off with suction and recrystallized in ethanol.

M.p.: 113.5-115.5° C.

### I1. 3-(2,2-Difluoro-ethoxy)-4-methoxy-benzaldehyde

10.04 g of isovanillin and 15.5 g of potassium carbonate are placed in an autoclave. 50 ml of DMF are added as well as 12.44 g of 2-bromo-1,1-difluoroethane. The autoclave is closed and heated at 60° C. for 20 h. Then the solids are filtered off and washed with 120 ml of DMF. About 120 ml of he solvent are distilled off and the residue poured on 200 ml of ice/water, where the product preciptates. After stirring the slurry for 30 minutes the product is filtered off and dried to give 13.69 g of the desired product.

Commercial Utility

The compounds according to the invention have useful pharmacological properties which make them industrially utilizable. As selective cyclic nucleotide phosphodiesterase (PDE) inhibitors (specifically of type 4), they are suitable on the one hand as bronchial therapeutics (for the treatment of airway obstructions on account of their dilating action but also on account of their respiratory rate- or respiratory drive-increasing action) and for the removal of erectile dysfunction on account of their vascular dilating action, but on the other hand especially for the treatment of disorders, in particular of an inflammatory nature, e.g. of the airways (asthma prophylaxis), of the skin, of the intestine, of the eyes, of the CNS and of the joints, which are mediated by mediators such as histamine, PAF (platelet-activating factor), arachidonic acid derivatives such as leukotrienes and prostaglandins, cytokines, interleukins, chemokines, alpha-, beta- and gamma-interferon, tumor necrosis factor (TNF) or oxygen free radicals and proteases. In this context, the compounds according to the invention are distinguished by a low toxicity, a good enteral absorption (high bioavailability), a large therapeutic breadth and the absence of significant side effects.

On account of their PDE-inhibiting properties, the compounds according to the invention can be employed in human and veterinary medicine as therapeutics, where they can be used, for example, for the treatment and prophylaxis of the following illnesses: acute and chronic (in particular inflammatory and allergen-induced) airway disorders of varying origin (bronchitis, allergic bronchitis, bronchial asthma, emphysema, COPD); dermatoses (especially of proliferative, inflammatory and allergic type) such as psoriasis (vulgaris), toxic and allergic contact eczema, atopic eczema, seborrhoeic eczema, Lichen simplex, sunburn, pruritus in the anogenital area, alopecia areata, hypertrophic scars, discoid lupus erythematosus, follicular and widespread pyodermias, endogenous and exogenous acne, acne rosacea and other proliferative, inflammatory and allergic skin disorders; disorders which are based on an excessive release of TNF and leukotrienes, for example disorders of the arthritis type (rheumatoid arthritis, rheumatoid spondylitis, osteoarthritis and other arthritic conditions), disorders of the immune system (AIDS, multiple sclerosis), graft versus host reaction, allograft rejections, types of shock (septic shock, endotoxin shock, gramnegative sepsis, toxic shock syndrome and ARDS (adult respiratory distress syndrome)) and also generalized inflammations in the gastrointestinal region (Crohn's disease and ulcerative colitis); disorders which are based on allergic and/or chronic, immunological false reactions in the region of the upper airways (pharynx, nose) and the adjacent regions (paranasal sinuses, eyes), such as allergic rhinitis/sinusitis, chronic rhinitis/sinusitis, allergic conjunctivitis and also nasal polyps; but also disorders of the heart which can be treated by PDE inhibitors, such as cardiac insufficiency, or disorders which can be treated on account of the tissue-relaxant action of the PDE inhibitors, such as, for example, erectile dysfunction or colics of the kidneys and of the ureters in connection with kidney stones. In addition, the compounds of the invention are useful in the treatment of diabetes insipidus and conditions associated with cerebral metabolic inhibition, such as cerebral senility, senile dementia (Alzheimer's disease), memory impairment associated with Parkinson's disease or multiinfarct dementia; and also illnesses of the central nervous system, such as depressions or arteriosclerotic dementia; as well as for enhancing cognition. Yet in addition, the compounds of the invention are useful in the treatment of diabetes mellitus, leukaemia and osteoporosis.

The invention further relates to a method for the treatment of mammals, including humans, which are suffering from one of the above mentioned illnesses. The method is characterized in that a therapeutically active and pharmacologically effective and tolerable amount of one or more of the compounds according to the invention is administered to the ill mammal.

The invention further relates to the compounds according to the invention for use in the treatment and/or prophylaxis of illnesses, especially the illnesses mentioned.

The invention also relates to the use of the compounds according to the invention for the production of pharmaceutical compositions which are employed for the treatment and/or prophylaxis of the illnesses mentioned.

The invention also relates to the use of the compounds according to the invention for the production of pharmaceutical compositions for treating disorders which are mediated by phosphodiesterases, in particular PDE4-mediated disorders, such as, for example, those mentioned in the specification of this invention or those which are apparent or known to the skilled person.

The invention also relates to the use of the compounds according to the invention for the manufacture of pharmaceutical compositions having PDE4 inhibitory activity.

The invention furthermore relates to pharmaceutical compositions for the treatment and/or prophylaxis of the illnesses mentioned comprising one or more of the compounds according to the invention.

The invention yet furthermore relates to compositions comprising one or more compounds according to this invention and a pharmaceutically acceptable carrier. Said compositions can be used in therapy, such as e.g. for treating, preventing or ameliorating one or more of the abovementioned diseases.

The invention still yet furthermore relates to pharmaceutical compositions according to this invention having PDE, particularly PDE4, inhibitory activity.

Additionally, the invention relates to an article of manufacture, which comprises packaging material and a pharmaceutical agent contained within said packaging material, wherein the pharmaceutical agent is therapeutically effective for antagonizing the effects of the cyclic nucleotide phosphodiesterase of type 4 (PDE4), ameliorating the symptoms of an PDE4-mediated disorder, and wherein the packaging material comprises a label or package insert which indicates that the pharmaceutical agent is useful for preventing or treating PDE4-mediated disorders, and wherein said pharmaceutical agent comprises one or more compounds of formula 1 according to the invention. The packaging material, label and package insert otherwise parallel or resemble what is generally regarded as standard packaging material, labels and package inserts for pharmaceuticals having related utilities.

The pharmaceutical compositions are prepared by processes which are known per se and familiar to the person skilled in the art. As pharmaceutical compositions, the compounds according to the invention (=active compounds) are either employed as such, or preferably in combination with suitable pharmaceutical auxiliaries and/or excipients, e.g. in the form of tablets, coated tablets, capsules, caplets, suppositories, patches (e.g. as TTS), emulsions, suspensions, gels or solutions, the active compound content advantageously being between 0.1 and 95% and where, by the appropriate choice of the auxiliaries and/or excipients, a pharmaceutical administration form (e.g. a delayed release form or an enteric form) exactly suited to the active compound and/or to the desired onset of action can be achieved.

The person skilled in the art is familiar with auxiliaries, excipients, carriers, vehicles, diluents or adjuvants which are suitable for the desired pharmaceutical formulations on account of his/her expert knowledge. In addition to solvents, gel formers, ointment bases and other active compound excipients, for example antioxidants, dispersants, emulsifiers, preservatives, solubilizers, colorants, complexing agents or permeation promoters, can be used.

The administration of the pharmaceutical compositions according to the invention may be performed in any of the generally accepted modes of administration available in the art. IIlustrative examples of suitable modes of administration include intravenous, oral, nasal, parenteral, topical, transdermal and rectal delivery. Oral delivery is preferred.

For the treatment of disorders of the respiratory tract, the compounds according to the invention are preferably also administered by inhalation in the form of an aerosol; the aerosol particles of solid, liquid or mixed composition preferably having a diameter of 0.5 to 10 μm, advantageously of 2 to 6 μm.

Aerosol generation can be carried out, for example, by pressure-driven jet atomizers or ultrasonic atomizers, but advantageously by propellant-driven metered aerosols or propellant-free administration of micronized active compounds from inhalation capsules.

Depending on the inhaler system used, in addition to the active compounds the administration forms additionally contain the required excipients, such as, for example, propellants (e.g. Frigen in the case of metered aerosols), surface-active substances, emulsifiers, stabilizers, preservatives, flavorings, fillers (e.g. lactose in the case of powder inhalers) or, if appropriate, further active compounds.

For the purposes of inhalation, a large number of apparatuses are available with which aerosols of optimum particle size can be generated and administered, using an inhalation technique which is as right as possible for the patient. In addition to the use of adaptors (spacers, expanders) and pear-shaped containers (e.g. Nebulator®, Volumatic®), and automatic devices emitting a puffer spray (Autohaler®), for metered aerosols, in particular in the case of powder inhalers, a number of technical solutions are available (e.g. Diskhaler®, Rotadisk®, Turbohaler® or the inhaler described in European Patent Application EP 0 505 321), using which an optimal administration of active compound can be achieved.

For the treatment of dermatoses, the compounds according to the invention are in particular administered in the form of those pharmaceutical compositions which are suitable for topical application. For the production of the pharmaceutical compositions, the compounds according to the invention (=active compounds) are preferably mixed with suitable pharmaceutical auxiliaries and further processed to give suitable pharmaceutical formulations. Suitable pharmaceutical formulations are, for example, powders, emulsions, suspensions, sprays, oils, ointments, fatty ointments, creams, pastes, gels or solutions.

The pharmaceutical compositions according to the invention are prepared by processes known per se. The dosage of the active compounds is carried out in the order of magnitude customary for PDE inhibitors. Topical application forms (such as ointments) for the treatment of dermatoses thus contain the active compounds in a concentration of, for example, 0.1-99%. The dose for administration by inhalation is customarly between 0.01 and 3 mg per day. The customary dose in the case of systemic therapy (p.o. or i.v.) is between 0.003 and 3 mg/kg per day. In another embodiment, the dose for administration by inhalation is between 0.1 and 3 mg per day, and the dose in the case of systemic therapy (p.o. or i.v.) is between 0.03 and 3 mg/kg per day.

Biological Investigations

The second messenger cyclic AMP (CAMP) is well-known for inhibiting inflammatory and immunocompetent cells. The PDE4 isoenzyme is broadly expressed in cells involved in the initiation and propagation of inflammatory diseases (H Tenor and C Schudt, in “Phosphodiesterase Inhibitors”, 21-40, “The Handbook of Immunopharmacology”, Academic Press, 1996), and its inhibition leads to an increase of the intracellular cAMP concentration and thus to the inhibition of cellular activation (J E Souness et al., Immunopharmacology 47: 127-162, 2000).

The antiinflammatory potential of PDE4 inhibitors in vivo in various animal models has been described (M M Teixeira, TiPS 18: 164-170, 1997). For the investigation of PDE4 inhibition on the cellular level (in vitro), a large variety of proinflammatory responses can be measured. Examples are the superoxide production of neutrophilic (C Schudt et al., Arch Pharmacol 344: 682-690, 1991) or eosinophilic (A Hatzelmann et al., Brit J Pharmacol 114: 821-831, 1995) granulocytes, which can be measured as luminol-enhanced chemiluminescence, or the synthesis of tumor necrosis factor-α in monocytes, macrophages or dendritic cells (Gantner et al., Brit J Pharmacol 121: 221-231, 1997, and Pulmonary Pharmacol Therap 12: 377-386, 1999). In addition, the immunomodulatory potential of PDE4 inhibitors is evident from the inhibition of T-cell responses like cytokine synthesis or proliferation (D M Essayan, Biochem Pharmacol 57: 965-973, 1999). Substances which inhibit the secretion of the afore-mentioned proinflammatory mediators are those which inhibit PDE4. PDE4 inhibition by the compounds according to the invention is thus a central indicator for the suppression of inflammatory processes.

Methods for Measuring Inhibition of PDE4 Activity

The PDE4B2 (GB no. M97515) was a gift of Prof. M. Conti (Stanford University, USA). It was amplified from the original plasmid (pCMV5) via PCR with primers. Rb9 (5′- GCCAGCGTGCAAATAATGAAGG -3′) and Rb 10 (5′- AGAGGGGGATTATGTATCCAC -3′) and cloned into the pCR-Bac vector (Invitrogen, Groningen, NL).

The recombinant baculovirus was prepared by means of homologous recombination in SF9 insect cells. The expression plasmid was cotransfected with Bac-N-Blue (Invitrogen, Groningen, NL) or Baculo-Gold DNA (Pharmingen, Hamburg) using a standard protocol (Pharmingen, Hamburg). Wt virus-free recombinant virus supernatant was selected using plaque assay methods. After that, high-titre virus supernatant was prepared by amplifying 3 times. PDE was expressed in SF21 cells by infecting 2×10⁶ cells/ml with an MOI (multiplicity of infection) between 1 and 10 in serum-free SF900 medium (Life Technologies, Paisley, UK). The cells were cultured at 28° C. for 48-72 hours, after which they were pelleted for 5-10 min at 1000 g and 4° C.

The SF21 insect cells were resuspended, at a concentration of approx. 10⁷ cells/ml, in ice-cold (4° C.) homogenization buffer (20 mM Tris, pH 8.2, containing the following additions: 140 mM NaCl, 3.8 mM KCl, 1 mM EGTA, 1 mM MgCl₂, 10 mM β-mercaptoethanol, 2 mM benzamidine, 0.4 mM Pefablock, 10 μM leupeptin, 10 μM pepstatin A, 5 μM trypsin inhibitor) and disrupted by ultrasonication. The homogenate was then centrifuged for 10 min at 1000×g and the supernatant was stored at −80° C. until subsequent use (see below). The protein content was determined by the Bradford method (BioRad, Munich) using BSA as the standard.

PDE4B2 activity is inhibited by the said compounds in a modified SPA (scintillation proximity assay) test, supplied by Amersham Biosciences (see procedural instructions “phosphodiesterase [3H]cAMP SPA enzyme assay, code TRKQ 7090”), carried out in 96-well microtitre plates (MTP's). The test volume is 100 μl and contains 20 mM Tris buffer (pH 7.4), 0.1 mg of BSA (bovine serum albumin)/ml, 5 mM Mg²⁺, 0.5 μM cAMP (including about 50,000 cpm of [3H]cAMP), 1 μl of the respective substance dilution in DMSO and sufficient recombinant PDE (1000×g supernatant, see above) to ensure that 10-20% of the cAMP is converted under the said experimental conditions. The final concentration of DMSO in the assay (1% v/v) does not substantially affect the activity of the PDE investigated. After a preincubation of 5 min at 37° C., the reaction is started by adding the substrate (cAMP) and the assay is incubated for a further 15 min; after that, it is stopped by adding SPA beads (50 μl). In accordance with the manufacturer's instructions, the SPA beads had previously been resuspended in water, but were then diluted 1:3 (v/v) in water; the diluted solution also contains 3 mM IBMX to ensure a complete PDE activity stop. After the beads have been sedimented (>30 min), the MTP's are analyzed in commercially available luminescence detection devices. The corresponding IC₅₀ values of the compounds for the inhibition of PDE activity are determined from the concentration-effect curves by means of non-linear regression.

Representative inhibitory values determined for the compounds according to the invention follow from the following table A, in which the numbers of the compounds correspond to the numbers of the Examples.

TABLE A
Inhibition of the PDE4 activity

| Compound | −log IC₅₀(mol/l) |
|---|---|
| 19 | 8.83 |
| 1 to 12, and 14 to 17 | The inhibitory values of these mentioned |
| | Examples lie in the range from 8.42 to 9.73 |

1. A compound of formula I, 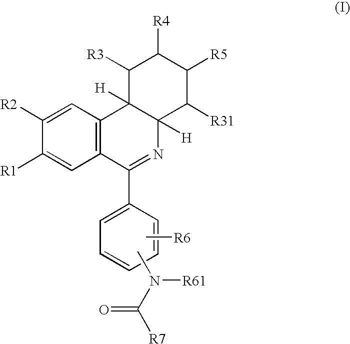
in which
R1 is hydroxyl, 1-4C-alkoxy, 3-7C-cycloalkoxy, 3-7C-cycloalkylmethoxy, 2,2-difluoroethoxy, or completely or predominantly fluorine-substituted 1-4C-alkoxy,
R2 is hydroxyl, 1-4C-alkoxy, 3-7C-cycloalkoxy, 3-7C-cycloalkylmethoxy, 2,2-difluoroethoxy, or completely or predominantly fluorine-substituted 1-4C-alkoxy,
or in which
R1 and R2 together are a 1-2C-alkylenedioxy group,
R3 is hydrogen or 1-4C-alkyl,
R31 is hydrogen or 1-4C-alkyl,
wherein either, in a first embodiment (embodiment a)
R4 is —O—R41, in which
R41 is hydrogen, 1-4C-alkyl, 1-4C-alkoxy-1-4C-alkyl, hydroxy-2-4C-alkyl, 1-7C-alkylcarbonyl, or completely or predominantly fluorine-substituted 1-4C-alkyl, and
R5 is hydrogen or 1-4C-alkyl,
or, in a second embodiment (embodiment b)
R4 is hydrogen or 1-4C-alkyl, and
R5 is —O—R51, in which
R51 is hydrogen, 1-4C-alkyl, 1-4C-alkoxy-1-4C-alkyl, hydroxy-2-4C-alkyl, 1-7C-alkylcarbonyl, or completely or predominantly fluorine-substituted 1-4C-alkyl,
R6 is hydrogen, halogen, 1-4C-alkyl or 1-4C-alkoxy,
R61 is hydrogen, 1-4C-alkyl or 1-4C-alkoxy-2-4C-alkyl,
R7 is Het1, Har1, 3-7C-cycloalkyl, or 1-4C-alkyl substituted by R8, in which
Het1 is optionally substituted by R71 and is a monocyclic monocylic 3- to 7-membered fully saturated heterocyclic ring radical,
which is bonded via a ring carbon atom to the carbonyl moiety of the —C(O)N(R61)- group, and
which comprises one nitrogen atom and optionally one further heteroatom selected from the group consisting of nitrogen, oxygen and sulfur, and, optionally,
to which ring one or two oxo groups are bonded, in which
R71 is 1-4C-alkyl, or completely or partially fluorine-substituted 1-4C-alkyl,
Har1 is optionally substituted by R72 and/or R73, and is a 5- or 6-membered monocyclic unsaturated heteroaryl radical comprising 1 to 4 heteroatoms independently selected from the group consisting of oxygen, nitrogen and sulfur, in which
R72 is halogen, 1-4C-alkyl, 1-4C-alkoxy, 1-4C-alkoxy-2-4C-alkoxy, 1-4C-alkylthio, cyano, 1-4C-alkoxycarbonyl, carboxyl, hydroxyl, -A-N(R721)R722, pyridyl, or completely or partially fluorine-substituted 1-4C-alkyl, in which
A is a bond or 1-4C-alkylene,
R721 is hydrogen or 1-4C-alkyl,
R722 is hydrogen or 1-4C-alkyl,
or R721 and R722 together and with inclusion of the nitrogen atom, to which they are attached, form a heterocyclic ring Het2, in which
Het2 is optionally substituted by R723, and is a 3- to 7-membered saturated or unsaturated monocyclic heterocyclic ring radical comprising the nitrogen atom, to which R721 and R722 are bonded, and optionally one to three further heteroatoms independently selected from the group consisting of oxygen, nitrogen and sulfur, in which
R723 is 1-4C-alkyl,
R73 is halogen, 1-4C-alkoxy, 1-4C-alkoxy-2-4C-alkoxy, 1-4C-alkylthio, hydroxyl, amino or mono- or di-1-4C-alkylamino,
R8 is 1-4C-alkoxy, carbamoyl, carboxyl, 1-4C-alkoxycarbonyl, mono- or di-1-4C-alkylaminocarbonyl or —N(R81)R82, in which
R81 is hydrogen, 1-4C-alkyl, carbamoyl, amidino or 1-4C-alkylcarbonyl,
R82 is hydrogen or 1-4C-alkyl,
or R81 and R82 together and with inclusion of the nitrogen atom, to which they are attached, form a heterocyclic ring Het3, in which
Het3 is optionally substituted by R811, and is a 3- to 7-membered saturated monocyclic heterocyclic ring radical comprising the nitrogen atom, to which R81 and R82 are bonded, and optionally one further heteroatom selected from the group consisting of oxygen, nitrogen and sulfur, in which
R811 is 1-4C-alkyl,
or an enantiomer, salt, N-oxide, salt of an N-oxide or enantiomer of an N-oxide thereof.

2. A compound of formula I according to claim 1 in which
R1 is 1-2C-alkoxy, 3-5C-cycloalkoxy, 3-5C-cycloalkylmethoxy, 2,2-difluoroethoxy, or completely or predominantly fluorine-substituted 1-2C-alkoxy,
R2 is 1-2C-alkoxy, 3-5C-cycloalkoxy, 3-5C-cycloalkylmethoxy, 2,2-difluoroethoxy, or completely or predominantly fluorine-substituted 1-2C-alkoxy,
R3 is hydrogen,
R31 is hydrogen,
wherein either, in a first embodiment (embodiment a)
R4 is —O—R41, in which
R41 is hydrogen or 1-4C-alkylcarbonyl, and
R5 is hydrogen,
or, in a second embodiment (embodiment b),
R4 is hydrogen, and
R5 is —O—R51, in which
R51 is hydrogen or 1-4C-alkylcarbonyl,
R6 is hydrogen,
R61 is hydrogen,
R7 is Het1, Har1, 3-7C-cycloalkyl, or 1-4C-alkyl substituted by R8, in which
Het1 is optionally substituted by R71 and is a monocyclic 5- to 7-membered fully saturated heterocyclic ring radical, which is bonded via a ring carbon atom to the carbonyl moiety of the —C(O)N(R61)- group, and which comprises one nitrogen atom and optionally one further heteroatom selected from the group consisting of nitrogen, oxygen and sulfur, and, optionally, to which ring one or two oxo groups are bonded, in which
R71 is 1-4C-alkyl, or completely or partially fluorine-substituted 1-4C-alkyl,
Har1 is optionally substituted by R72 and/or R73, and is either a 6-membered monocyclic unsaturated heteroaryl radical comprising one or two nitrogen atoms, or a 5-membered monocyclic unsaturated heteroaryl radical comprising 1 to 4 heteroatoms independently selected from the group consisting of oxygen, nitrogen and sulfur, in which
R72 is halogen, 1-4C-alkyl, 1-4C-alkoxy, 1-4C-alkoxy-2-4C-alkoxy,1-4C-alkylthio, cyano, 1-4C-alkoxycarbonyl, carboxyl, hydroxyl, -A-N(R721)R722, pyridyl, or completely or partially fluorine-substituted 1-4C-alkyl, in which
A is a bond or 1-4C-alkylene,
R721 is hydrogen or 1-4C-alkyl,
R722 is hydrogen or 1-4C-alkyl,
or R721 and R722 together and with inclusion of the nitrogen atom, to which they are attached, form a heterocyclic ring Het2, in which
Het2 is optionally substituted by R723, and is a 3- to 7-membered saturated or unsaturated monocyclic heterocyclic ring radical comprising the nitrogen atom, to which R721 and R722 are bonded, and optionally one to three further heteroatoms independently selected from the group consisting of oxygen, nitrogen and sulfur, in which
R723 is 1-4C-alkyl,
R73 is halogen, 1-4C-alkoxy, 1-4C-alkoxy-2-4C-alkoxy, 1-4C-alkylthio, hydroxyl, amino or mono- or di-1-4C-alkylamino,
R8 is 1-4C-alkoxy, carbamoyl, mono- or di-1-4C-alkylaminocarbonyl or —N(R81)R82, in which
R81 is hydrogen, 1-4C-alkyl, carbamoyl, amidino or 1-4C-alkylcarbonyl,
R82 is hydrogen or 1-4C-alkyl,
or R81 and R82 together and with inclusion of the nitrogen atom, to which they are attached, form a heterocyclic ring Het3, in which
Het3 is optionally substituted by R811, and is a 3- to 7-membered saturated monocyclic heterocyclic ring radical comprising the nitrogen atom, to which R81 and R82 are bonded, and optionally one further heteroatom selected from the group consisting of oxygen, nitrogen and sulfur, in which
R811 is 1-4C-alkyl,
or an enantiomer, salt, N-oxide, salt of an N-oxide or enantiomer of an N-oxide thereof.

3. A compound of formula I according to claim 1 in which
R1 is 1-2C-alkoxy, 3-5C-cycloalkoxy, 3-5C-cycloalkylmethoxy, 2,2-difluoroethoxy, or completely or predominantly fluorine-substituted 1-2C-alkoxy,
R2 is 1-2C-alkoxy, 3-5C-cycloalkoxy, 3-5C-cycloalkylmethoxy, 2,2-difluoroethoxy, or completely or predominantly fluorine-substituted 1-2C-alkoxy,
R3 is hydrogen,
R31 is hydrogen,
R4 is —O—R41, in which
R41 is 1-4C-alkylcarbonyl or hydrogen,
R5 is hydrogen,
R6 is hydrogen,
R61 is hydrogen,
R7 is Het1, Har1, 3-7C-cycloalkyl, or 1-4C-alkyl substituted by R8, in which
Het1 is optionally substituted by R71 and is a monocyclic 5- to 7-membered fully saturated heterocyclic ring radical,
which is bonded via a ring carbon atom to the carbonyl moiety of the —C(O)N(R61)- group, and
which comprises one nitrogen atom, and, optionally, to which ring one oxo group is bonded, in which
R71 is 1-4C-alkyl, or completely or partially fluorine-substituted1-2C-alkyl,
Har1 is optionally substituted by R72 and/or R73, and is a 6-membered monocyclic unsaturated heteroaryl radical comprising one or two nitrogen atoms, in which
R72 is halogen, 1-4C-alkoxy, 1-4C-alkoxy-ethoxy, 1-2C-alkylthio, hydroxyl, amino or mono- or di-1-2C-alkylamino,
R73 is halogen, 1-4C-alkoxy, 1-2C-alkoxy-ethoxy, 1-2C-alkylthio, hydroxyl, amino or mono- or di-1-2C-alkylamino,
R8 is 1-4C-alkoxy, carbamoyl, mono- or di-1-4C-alkylaminocarbonyl or —N(R81)R82, in which
R81 is hydrogen, 1-4C-alkyl, carbamoyl or 1-4C-alkylcarbonyl,
R82 is hydrogen or 1-4C-alkyl,
or R81 and R82 together and with inclusion of the nitrogen atom, to which they are attached, form a heterocyclic ring Het3, in which
Het3 is optionally substituted by R811, and is a 3- to 7-membered saturated monocyclic heterocyclic ring radical comprising the nitrogen atom, to which R81 and R82 are bonded, and optionally one further heteroatom selected from the group consisting of oxygen, nitrogen and sulfur, in which
R811 is 1-2C-alkyl,
or an enantiomer, salt, N-oxide, salt of an N-oxide or enantiomer of an N-oxide thereof.

4. A compound of formula I according to claim 1 in which
R1 is 1-2C-alkoxy, 2,2-difluoroethoxy, or completely or predominantly fluorine-substituted 1-2C-alkoxy,
R2 is 1-2C-alkoxy, 2,2-difluoroethoxy, or completely or predominantly fluorine-substituted 1-2C-alkoxy,
R3 is hydrogen,
R31 is hydrogen,
R4 is —O—R41, in which
R41 is hydrogen,
R5 is hydrogen,
R6 is hydrogen,
R61 is hydrogen,
R7 is Het1, Har1, 3-5C-cycloalkyl, or 1-4C-alkyl substituted by R8, in which
Het1 is 1-N—(R71)-piperdin-4-yl or pyrrolidin-2-on-5-yl, in which
R71 is 1-4C-alkyl,
Har1 is optionally substituted by R72 and/or R73, and is pyridinyl, in which
R72 is 1-4C-alkoxy,
R73 is 1-4C-alkoxy,
R8 is 1-4C-alkoxy, carbamoyl, mono- or di-1-4C-alkylaminocarbonyl or —N(R81)R82, in which
R81 is 1-4C-alkyl, carbamoyl or 1-4C-alkylcarbonyl,
R82 is hydrogen or 1-4C-alkyl,
or R81 and R82 together and with inclusion of the nitrogen atom, to which they are attached, form a heterocyclic ring Het3, in which
Het3 is piperidin-1-yl, pyrrolidin-1-yl, 4-N—(R811)-piperazin-1-yl, 4-N—(R81l)-homopiperazin-1-yl, homopiperidin-1-yl, morpholin-4-yl or thiomorpholin-4-yl, in which
R811 is 1-2C-alkyl,
or an enantiomer, salt, N-oxide, salt of an N-oxide or enantiomer of an N-oxide thereof

5. A compound of formula I according to claim 1 in which
R1 is 1-2C-alkoxy, 2,2-difluoroethoxy, or completely or predominantly fluorine-substituted 1-2C-alkoxy,
R2 is 1-2C-alkoxy, 2,2-difluoroethoxy, or completely or predominantly fluorine-substituted1-2C-alkoxy,
R3 is hydrogen,
R31 is hydrogen,
R4 is —O—R41, in which
R41 is hydrogen,
R5 is hydrogen,
R6 is hydrogen,
R61 is hydrogen,
R7 is Het1, Har1, 3-5C-cycloalkyl, or 1-4C-alkyl substituted by R8, in which
Het1 is 1-N—(R71)-piperdin-4-yl or pyrrolidin-2-on-5-yl, in which
R71 is 1-4C-alkyl,
Har1 is 2,6-dimethoxypyridin-3-yl,
R8 is 1-4C-alkoxy, carbamoyl, mono- or di-1-4C-alkylaminocarbonyl or —N(R81)R82, in which
R81 is 1-4C-alkyl, carbamoyl or 1-4C-alkylcarbonyl,
R82 is hydrogen or 1-4C-alkyl,
or R81 and R82 together and with inclusion of the nitrogen atom, to which they are attached, form a heterocyclic ring Het3, in which
Het3 is piperidin-1-yl,
or an enantiomer, salt, N-oxide, salt of an N-oxide or enantiomer of an N-oxide thereof.

6. A compound of formula I according to claim 1 in which
R1 is methoxy or ethoxy,
R2 is methoxy, ethoxy, difluoromethoxy or 2,2-difluoroethoxy,
R3 is hydrogen,
R31 is hydrogen,
R4 is —O—R41, in which
R41 is hydrogen,
R5 is hydrogen,
R6 is hydrogen,
R61 is hydrogen,
R7 is Het1, Har1, cyclopropyl, or 1-4C-alkyl substituted by R8, in which
Het1 is 1-N—(R71)-piperdin-4-yl or pyrrolidin-2-on-5-yl, in which
R71 is methyl,
Har1 is 2,6-dimethoxypyridin-3-yl,
R8 is methoxy, carbamoyl, dimethylaminocarbonyl or —N(R81)R82, in which
R81 is methyl, carbamoyl or acetyl,
R82 is hydrogen or methyl,
or R81 and R82 together and with inclusion of the nitrogen atom, to which they are attached, form a heterocyclic ring Het3, in which
Het3 is piperidin-1-yl,
or an enantiomer, salt, N-oxide, salt of an N-oxide or enantiomer of an N-oxide thereof.

7. A compound of formula I according to claim 1 comprising one or more of the following:
R1 is methoxy,
R2 is methoxy, ethoxy, difluoromethoxy or 2,2-difluoroethoxy, and
R3 and R31 are hydrogen,
R4 is —O—R41, in which
R41 is hydrogen, and
R5 is hydrogen,
R6 is hydrogen,
R61 is hydrogen,
the radical R7C(O)N(R61)- is bonded to the meta or para position with respect to the binding position at which the phenyl group is bonded or an enantiomer, salt, N-oxide, salt of an N-oxide or enantiomer of an N-oxide thereof.

8. A compound of formula I according to claim 1 selected from the group consisting of
N-[3-((2RS,4aRS,10bRS)-9-Ethoxy-2-hydroxy-8-methoxy-1,2,3,4,4a,10b-hexahydro-phenanthridin-6-yl)-phenyl]-2-methoxy-acetamide,
N-[4-((2RS,4aRS,10bRS)-9-Ethoxy-2-hydroxy-8-methoxy-1,2,3,4,4a,10b-hexahydro-phenanthridin-6-yl)-phenyl]-3-methoxy-propionamide,
Cyclopropanecarboxylic acid [3-((2RS,4aRS,10bRS)-9-ethoxy-2-hydroxy-8-methoxy-1,2,3,4,4a,10b-hexahydro-phenanthridin-6-yl)-phenyl]-amide,
N-[4-((2RS,4aRS,10bRS)-9-Ethoxy-2-hydroxy-8-methoxy-1,2,3,4,4a,10b-hexahydro-phenanthridin-6-yl)-phenyl]-3-piperidin-1-yl-propionamide,
N-[4-((2RS,4aRS,10bRS)-9-Ethoxy-2-hydroxy-8-methoxy-1,2,3,4,4a,10b-hexahydro-phenanthridin-6-yl)-phenyl]-N′,N′-dimethyl-succinamide,
1-Methyl-piperidine-4-carboxylic acid [4-((2RS,4aRS, 10bRS)-9-ethoxy-2-hydroxy-8-methoxy-1,2,3,4,4a,10b-hexahydro-phenanthridin-6-yl)-phenyl]-amide,
Cyclopropanecarboxylic acid [4-((2RS,4aRS,10bRS)-9-ethoxy-2-hydroxy-8-methoxy-1,2,3,4,4a,10b-hexahydro-phenanthridin-6-yl)-phenyl]-amide,
N-[3-((2RS,4aRS,10bRS)-9-Ethoxy-2-hydroxy-8-methoxy-1,2,3,4,4a,10b-hexahydro-phenanthridin-6-yl)-phenyl]-3-methoxy-propionamide,
N-[4-((2RS,4aRS,10bRS)-9-Ethoxy-2-hydroxy-8-methoxy-1,2,3,4,4a,10b-hexahydro-phenanthridin-6-yl)-phenyl]-2-methoxy-acetamide,
N-[3-((2RS,4aRS,10bRS)-9-Ethoxy-2-hydroxy-8-methoxy-1,2,3,4,4a,10b-hexahydro-phenanthridin-6-yl)-phenyl]-3-piperidin-1-yl-propionamide,
1-Methyl-piperidine-4-carboxylic acid [3-((2RS,4aRS,10bRS)-9-ethoxy-2-hydroxy-8-methoxy-1,2,3,4,4a,10b-hexahydro-phenanthridin-6-yl)-phenyl]-amide,
N-[3-((2RS,4aRS,10bRS)-9-Ethoxy-2-hydroxy-8-methoxy-1,2,3,4,4a,10b-hexahydro-phenanthridin-6-yl)-phenyl]-N′,N′-dimethyl-succinamide,
Dimethylamino-N-[4-((2RS,4aRS,10bRS)-9-ethoxy-2-hydroxy-8-methoxy-1,2,3,4,4a,10b-hexahydro-phenanthridin-6-yl)-phenyl]-butyramide,
Dimethylamino-N-[3-((2RS,4aRS,10bRS)-9-ethoxy-2-hydroxy-8-methoxy-1,2,3,4,4a,10b-hexahydro-phenanthridin-6-yl)-phenyl]-butyramide,
2-Acetylamino-N-[3-((2RS,4aRS,10bRS)-9-ethoxy-2-hydroxy-8-methoxy-1,2,3,4,4a,10b-hexahydro-phenanthridin-6-yl)-phenyl]-acetamide,
5-Oxo-pyrrolidine-2-carboxylic acid [3-((2RS,4aRS,10bRS)-9-ethoxy-2-hydroxy-8-methoxy-1,2,3,4,4a,10b-hexahydro-phenanthridin-6-yl)-phenyl]-amide,
N-[3-((2RS,4aRS,10bRS)-9-Ethoxy-2-hydroxy-8-methoxy-1,2,3,4,4a,10b-hexahydro-phenanthridin-6-yl)-phenyl]-2,6-dimethoxy-nicotinamide,
(2RS,4aRS,10bRS)-6-[3-(3-carbamoyl-propanoylamino)-phenyl]-9-ethoxy-8-methoxy-1,2,3,4,4a,10b-hexahydro-phenanthridin-2-ol,
N-[3-((2RS,4aRS,10bRS)-9-Ethoxy-2-hydroxy-8-methoxy-1,2,3,4,4a,10b-hexahydro-phenanthridin-6-yl)-phenyl]-3-ureido-propionamide,
Cyclopropanecarboxylic acid [3-((3SR,4aRS,10bRS)-9-ethoxy-3-hydroxy-8-methoxy-1,2,3,4,4a,10b-hexahydro-phenanthridin-6-yl)-phenyl]-amide and
the enantiomers, N-oxides, salts of the N-oxides and enantiomers of the N-oxides thereof.

9. A compound of formula I according to claim 1 which has with respect to the positions 4a and 10b the configuration shown in formula I*: 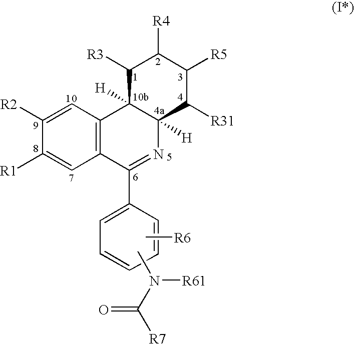
or a salt, N-oxide or salt of an N-oxide thereof.

10. A compound of formula I according to claim 1 which has with respect to the positions 2, 4a and 10b the configuration shown in formula Ia*****, or, which has with respect to the positions 3, 4a and 10b the configuration shown in formula Ib*****: 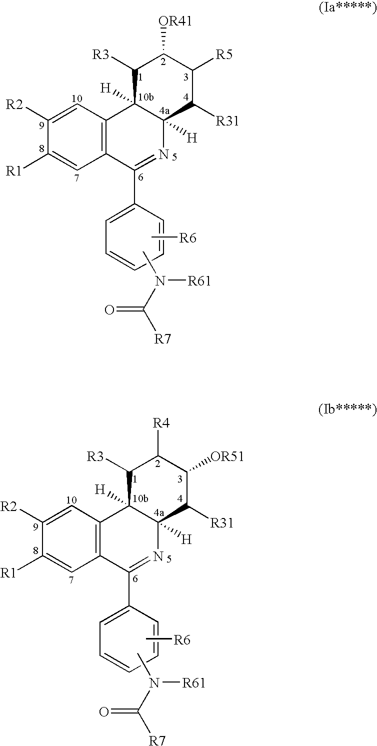
or a salt, N-oxide or salt of an N-oxide thereof.

11. (canceled)

12. A pharmaceutical composition comprising one or more compounds of formula I as claimed in claim 1, or a pharmaceutically acceptable salt, enantiomer, N-oxide, salt of an N-oxide or enantiomer of an N-oxide thereof, together with a pharmaceutically acceptable excipient and/or vehicle.

13-14. (canceled)

15. A method for treating an illness in a patient comprising administering to said patient a therapeutically effective amount of a compound of formula I as claimed in claim 1, or a pharmaceutically acceptable salt, enantiomer, N-oxide, salt of an N-oxide or enantiomer of an N-oxide thereof.

16. A method for treating an airway disorder in a patient comprising administering to said patient a therapeutically effective amount of a compound of formula I as claimed in claim 1, or a pharmaceutically acceptable salt, enantiomer, N-oxide, salt of an N-oxide or enantiomer of an N-oxide thereof.

17. A method for treating a PDE-mediated disorder in a patient comprising administering to said patient a therapeutically effective amount of a compound of formula I as claimed in claim 1, or a pharmaceutically acceptable salt, enantiomer, N-oxide, salt of an N-oxide or enantiomer of an N-oxide thereof.
